(19) United States
(12) Patent Application Publication
(10) Pub. No.: US 2005/0013862 A1
(43) Pub. Date: Jan. 20, 2005

(54) Functional powders for oral delivery

(75) Inventors: Michael John Tobyn, Wiltshire (GB); John Nicholas Staniforth, Bath (GB); David Bradley Brook Simpson, Bath (GB)

Correspondence Address: DAVIDSON, DAVIDSON & KAPPEL, LLC, 485 SEVENTH AVENUE, 14TH FLOOR, NEW YORK, NY, 10018, US

(73) Assignee: Vectura Limited, Chippenham (GB)

(21) Appl. No.: 10/487,633

(22) PCT Filed: Sep. 5, 2002
(86) PCT No.: PCT/IB02/04101

Related U.S. Application Data
(60) Provisional application No. 60/317,522, filed on Sep. 5, 2001.

(51) Int. Cl. A61K 9/24; A61K 9/14

(52) U.S. Cl. 424/472

(57) ABSTRACT

In certain embodiments the invention is directed to a drug formulation for gastrointestinal deposition comprising a non-compressed free flowing plurality of particles comprising a core comprising a drug and a pharmaceutically acceptable excipient, said core overcoated with a functional coating, said drug particles having a mean diameter of greater than 10 μm to about 1 mm.

This application claims the benefit of U.S. Provisional Ser. No. 60/317,522, filed Sep. 5, 2001, the entire disclosure of which is hereby incorporated by reference.

### FIELD OF THE INVENTION

The present is directed to a functional powders for oral use. Preferably, the powders are used in a multiple dose delivery device which dispenses a unit dose of the powder upon actuation.

### BACKGROUND OF THE INVENTION

The most prominent mode of delivery of therapeutic agents is by the oral route by means of solid dosage forms such as tablets and capsules. Oral administration of solid dosage forms is more convenient and accepted than other modes of administration, e.g. parenteral administration. However, the manufacture, dispensing and administration of solid dosage forms are not without associated problems and drawbacks.

With the manufacture of solid dosage forms, in addition to the active agent, it is necessary to combine other ingredients in the formulations for various reasons, such as to enhance physical appearance, to provide necessary bulk for tableting or capsuling, to improve stability, to improve compressibility or to aid in disintegration after administration. However, these added excipients have been shown to adversely influence the release, stability and bioavailability of the active ingredient. The added excipients are a particular problem with drugs which require a high dose in order to provide a therapeutic effect, e.g., biphosphonate drugs. The inclusion of the additional excipient can make the final tablet extremely large which could result in esophogeal damage due to the physical characteristics of the dosage form if it is not swallowed properly. Esophogeal damage can also be caused by toxicity caused by the drug itself, if the tablet becomes lodged in the throat or has an increased transit time through the esophagus, due to its increased size.

Further, the tableting of certain drugs has many associated production problems. In particular, many drugs, e.g., acetaminophen, have poor compressibility and cannot be directly compressed into solid dosage forms. Consequently, such drugs must either be wet granulated or manufactured in a special grade in order to be tableted which increases manufacturing steps and production costs.

The adherence to good manufacturing practices and process controls is essential in order to minimize dosage form to dosage form and batch to batch variations of the final product. Even strict adherence to these practices still is not a guarantee that acceptable variation will occur.

With the high cost of industrial scale production and governmental approval of solid dosage forms, such formulations are often available in a limited number of strengths, which only meet the needs of the largest sectors of the population. Unfortunately, this practice leaves many patients without acceptable means of treatment and physicians in a quandary with respect to individualizing dosages to meet the clinical needs of their patients.

The dispensing of oral solid dosage forms also makes the formulations susceptible to degradation and contamination due to repackaging, improper storage and manual handling.

There are also many patients who are unable or unwilling to take conventional orally administered dosage forms. For some patients, the perception of unacceptable taste or mouth feel of a dose of medicine leads to a gag reflex action that makes swallowing difficult or impossible. Other patients, e.g., pediatric and geriatric patients, find it difficult to ingest typical solid oral dosage forms, e.g., due to tablet size.

Other patients, particularly elderly patients, have conditions such as achlorhydria which hinders the successful use of oral solid dosage forms. Achlorhydria is a condition wherein there is an abnormal deficiency or absence of free hydrochloric acid in the gastric secretions of the stomach. This condition hinders the disintegration and/or dissolution of oral solid dosage forms, particularly dosage forms with high or insoluble excipient payloads. Thus, as the present dosage form is in multiparticulate form, it does need to undergo disintegration and/or dissolution to the same extent as solid dosage forms.

Flavored solutions/suspensions of some therapeutic agents have been developed to facilitate the oral administration of oral agents to patients normally having difficulty ingesting conventional solid oral dosage forms. While liquid formulations are more easily administered to the problem patient, liquid/suspension formulations are not without their own significant problems and restrictions. The liquid dose amount is not as easily controlled compared with tablet and capsule forms and many therapeutic agents are not sufficiently stable in solution/suspension form. Indeed, most suspension type formulations are typically reconstituted by the pharmacist and then have a limited shelf life even under refrigerated conditions. Another problem with liquid formulations which is not as much a factor with tablets and capsules is the taste of the active agent. The taste of some therapeutic agents is so unacceptable that liquid formulations are not a viable option. Further, solution/suspension type formulations are typically not acceptable where the active agent must be provided with a protective coating, e.g. a taste masking coating or an enteric coating to protect the active agent from the strongly acidic conditions of the stomach.

Due to the disadvantages of known drug delivery discussed above (as well as other disadvantages) there exists a need in the art for the development of a multiparticulate formulation for facilitating delivery of a wide range of therapeutic agents for gastrointestinal deposition and which minimize pulmonary deposition of materials having undesirable or unknown pulmonary toxicology but which are approved for oral delivery. In preferred embodiments, the formulation contains minimal excipient and is used in a multiple dose delivery device which dispenses a unit dose of the powder upon actuation.

### OBJECTS OF THE INVENTION

It is an object of the invention to provide a multiparticulate formulation containing a therapeutic agent for gastrointestinal deposition.

It is an object of certain embodiments of the invention to provide a multiparticulate formulation having at single coating which aids in the functionality of the formulation.

It is an object of certain embodiments of the invention to provide a multiparticulate formulation having at least two coatings which aid in the functionality of the formulation.

It is an object of certain embodiments of the invention to provide a high load multiparticulate formulation with minimal use of excipient.

It is a further object of certain embodiments of the invention to provide a multiparticulate formulation with improved weight variability, from dose to dose and batch to batch.

It is a further object of certain embodiments of the invention to provide a multiparticulate formulation which has minimal change in cohesiveness in response to humidity change

It is a further object of certain embodiments of the invention to provide a multiparticulate formulation which has minimal potential for water coalescence on the surface of the particles.

It is a further object of certain embodiments of the invention to provide a multiparticulate formulation which has minimal static charge between the particles.

It is an object of certain embodiments of the invention to provide a coated multiparticulate formulation which provides a controlled or delayed release of the active agent contained therein.

It is an object of certain embodiments of the invention to provide a coated multiparticulate formulation which tastemasks the active agent therein.

It is an object of certain embodiments of the invention to provide a coated multiparticulate formulation which contains a salivary stimulant to facilitate the swallowing of a unit dose of the multiparticulates upon oral delivery.

It is an object of the certain embodiments of invention to provide a coated multiparticulate formulation which contains a texture modifier to improve mouthfeel upon oral delivery.

It is an object of certain embodiments of the invention to provide a coated multiparticulate formulation which has a desired particle range in order to minimize pulmonary aspiration of particles.

It is an object of certain embodiments of the invention to provide a coated multiparticulate formulation which has a desired particle range in order to improve functionality of a the formulation in a multiple unit dosing device which delivers a unit dose of the formulation for oral administration or delivery upon actuation.

It is an object of certain embodiments of the invention to provide a coated multiparticulate formulation which has improved performance when used in a multiple unit dosing device which delivers a unit dose of the formulation for oral administration or delivery upon actuation.

It is an object of certain embodiments of the invention to provide a coated multiparticulate formulation which when divided into unit doses (e.g. with the use of a multiple unit dosing device) has weight uniformity of the formulation which is within the acceptable range of the weight uniformity of equivalent dosage forms which are tablets or capsules. A detailed discussion of weight uniformity is found in the USP/NF 23/18 section 905, hereby incorporated by reference in its entirety for all purposes.

It is an object of certain embodiments of the invention to provide methods of preparation of the coated multiparticulate dosage form disclosed herein.

It is an object of certain embodiments of the invention to provide methods of preparation of the multiple unit delivery systems containing the coated multiparticulate dosage form disclosed herein.

It is an object of certain embodiments of the invention to provide methods of preparation of multiparticulate dosage forms having a desired particles range.

It is an object of certain embodiments of the invention to provide methods of administering an active agent comprising administering a coated multiparticulate dosage form disclosed herein.

It is an object of certain embodiments of the invention to provide methods of administering an active agent comprising administering a coated multiparticulate dosage form disclosed herein via the use of a multiple unit delivery systems.

The above objects of the invention, and others are achieved by virtue of the present invention, which in certain embodiments is directed to a drug formulation for gastrointestinal deposition comprising a non-compressed free flowing plurality of particles comprising a core comprising a drug, the core overcoated with a functional coating.

In certain embodiments, the invention is directed to a drug formulation for gastrointestinal deposition comprising a non-compressed free flowing plurality of particles comprising a core comprising a drug and a pharmaceutically acceptable excipient, said core overcoated with a functional coating.

In certain embodiments, the invention is directed to a drug delivery system for delivery of a drug for gastrointestinal deposition. The system comprises a multiple unit dosing device comprising a housing and an actuator, the device containing multiple doses of the multiparticulate formulation disclosed herein, the device upon actuation delivering a unit dose of the multiparticulates for gastrointestinal deposition, the multiparticulates having a mean particle size of greater than 10 μm and preferably less than about 1 mm in order to minimize pulmonary deposition of the multiparticulates and such that an effective dose of the drug cannot be delivered into the lower lung of a human patient. The drug delivery system can be used to administer the unit dose of multiparticulates into the oral cavity of the patient (in-vivo) or to dispense the unit dose into an intermediate receptacle (ex-vivo) for subsequent gastrointestinal deposition. Oral drug delivery systems and devices for oral powders are disclosed in PCT/IB01/00251, hereby incorporated by reference in its entirety for all purposes.

In certain embodiments, the invention provides a method of preparing a drug delivery system for delivering multiple doses of a drug for gastrointestinal deposition comprising preparing a multiparticulate drug formulation as disclosed herein in a manner wherein the drug particles when placed in the oral cavity and swallowed are deposited to the gastrointestinal tract and not deposited in any substantial amount to the lungs; and placing multiple unit doses of said drug formulation in a device which meters a single unit dose for delivery.

In certain embodiments, the invention provides a method of treating a patient in need of multiple doses of a drug for gastrointestinal deposition comprising preparing multiparticulates comprising drug particles as disclosed herein in a manner wherein the drug particles when placed in the oral cavity and swallowed are deposited to the gastrointestinal tract and not deposited in any substantial amount to the lungs; placing multiple unit doses of the multiparticulates in a device which meters a single unit dose for delivery; and either (a) administering the unit dose into the oral cavity of a patient or (b) dispensing the unit dose into an intermediate receptacle and thereafter administering the unit dose into the oral cavity of the patient.

In certain embodiments, the invention provides a drug formulation for gastrointestinal deposition comprising a non-compressed free flowing plurality of particles comprising a drug and a pharmaceutically acceptable excipient, the particles having a mean diameter of greater than 10 μm to about 1 mm.

In certain embodiments, the particles of the invention comprise at least about 40% drug; at least about 50% drug; at least about 60% drug; at least about 80% drug; or at least about 90% drug.

In certain embodiments, the invention provides a method for delivery of a drug comprising delivering the multiparticulates disclosed herein comprising drug particles via the use of a multiple unit dosing device comprising a housing and an actuator, the device upon actuation delivering a unit dose of the multiparticulates disclosed herein, and thereafter re-using said device to deliver additional unit doses of the multiparticulates at appropriate dosing intervals.

In certain embodiments of the invention, greater than about 80% of the unit dose is deposited in the gastrointestinal tract, preferably greater than about 90% or greater than about 95%, or greater than about 99% and most preferably, about 100% of the unit dose is deposited in the gastrointestinal tract.

In preferred embodiments of the invention, the unit dose comprises a discreet collection of multiparticulates. For purposes of the invention, a “discreet collection” means that the multiparticulates are in the form of a non-compressed free flowing unit and not dispersed in a cloud or mist, which effectively minimizes inhalation of the active agent into the lungs of the patient. The unit dose can be, e.g., from about 0.01 mg to about 1.5 g, depending on the dose of the active agent being delivered. For example, the unit dose can be from about 1 mg to about 100 mg or from about 10 mg to about 50 mg. Preferably, the unit dose is administered to the tongue, most preferably towards the front of the tongue behind the teeth, where it can be easily swallowed with or without the need for an additional fluid. However the invention does contemplate delivery to any portion of the tongue, taking into account, e.g., the taste sensations of different sections of the tongue and/or individual patient preference associated with comfort, e.g. mouth position.

In certain embodiments of the invention, the mean diameter of the drug particles is of a size which minimizes their capacity to be inhaled into the lower lung. Typically, the mean particle size of the drug particles (or agglomerates) is greater than 10 μm, preferably greater than about 50 μm or greater than about 75 μm. In certain embodiments of the invention, the mean particle size range of the drug particles is from about 100 μm to about 1 mm, preferably from about 50 μm to about 500 μm. In preferred embodiments, greater than 80% of the drug particles have the above disclosed diameter (not mean diameter), e.g. 80% of the drug particles have a diameter of greater than 10 μm, or a diameter of from about 100 μm to about 1 mm. In other embodiments, greater than about 90% of the drug particles have the above disclosed diameter.

In certain embodiments of the invention, the mean diameter of the drug particles does not vary by greater than about 20%, preferably not greater than about 15% and most preferably not greater than about 10%.

In certain embodiments of the invention, the multiparticulates comprise a pharmaceutically acceptable excipient. The excipient preferably does not comprise more than about 60% by weight of the formulation; more preferably not more than about 50%; more preferably not more than about 40% by weight by weight; more preferably not more than about 20% by weight multiparticulates by weight, and most preferably not more than about 10% by weight of the formulation.

In certain embodiments of the invention, the multiple doses of the drug formulation disclosed herein are contained in a reservoir. The reservoir can contain an amount of multiparticulates to provide any number of unit doses, e.g. from about 2 doses to about 400 doses. For ease in patient compliance, the reservoir has a sufficient quantity of to provide e.g. a days supply, a months supply or a years supply of doses, e.g. 30 or 365 for once daily dosing for a month or year, respectively.

In order to aid in patient compliance, certain embodiments of the invention include a counter or indicator to display the number of doses remaining in the system or the number of doses actuated.

In certain embodiments of the invention, the unit doses are individually metered prior to actuation, e.g., in the form of capsules or blisters, wherein each blister contains one individual unit dose. The system can be capable of containing any multiple of pre-metered unit doses, e.g. from about 2 to about 400 blisters.

The invention is also directed to methods of delivery (e.g., in vivo administration and ex vivo dispensing) and methods of treatment utilizing any of the disclosed embodiments directed to compositions of matter. The invention is also directed to methods of preparation of all of the disclosed embodiments.

The invention is also directed to methods of providing a therapeutic effect to a patient comprising administering to the patient a unit dose of a drug utilizing the systems and formulations disclosed herein. The invention is also directed to methods of preparing the systems and devices.

For purposes of the present invention, the term “device” refers to an apparatus capable of delivering a unit dose of drug.

The term “system” refers to a drug delivery device in combination with the disclosed multiparticulate drug having the specifications disclosed herein, e.g. drug particle size, excipient type, etc.

The term “discreet collection” refers to a non-compressed free flowing unit of multiparticulates with minimal particulate matter being dispersed in the surrounding environment (e.g., as a cloud or mist).

The term “drug” refers to any agent which is capable of providing a therapeutic effect to a patient upon gastrointestinal deposition. This encompasses all drugs which are intended for absorption for a systemic effect (regardless of their actual bioavailability) as well as drugs intended for a local effect in the gut and/or oral cavity, e.g. nystatin, antibiotics or local anesthetics.

The term “particle size” refers to the diameter of the particle.

The term “deposition” means the deposit of the unit dose at the intended point of absorption and/or action. For example, gastro-intestinal deposition means the intended deposit of the unit dose in the gastrointestinal system for e.g., absorption for a systemic effect or to exert a local effect. Pulmonary deposition means the intended deposit of drug into the lungs in order to provide a pharmaceutical effect, regardless that the unit dose may enter the oral cavity prior to pulmonary deposition.

The term “dispense”, when used in connection with the devices and systems of the present invention, means that the device or system delivers the unit dose ex vivo with the intent of subsequent administration to a mammal. For example, the device or system can dispense the unit dose into a food, a liquid, a spoon, or another intermediate receptacle.

The term “administer”, when used in connection with the devices and systems of the present invention, means that the device or system delivers the unit dose in vivo, i.e., directly into the gastrointestinal tract of a mammal.

The term “deliver” is meant to cover all ex vivo and in vivo delivery, i.e., dispensing and administering, respectively.

The term “patient” refers to humans as well as other mammals in need of a therapeutic agent, e.g., household pets or livestock. This term also refers to humans or mammals in need of or receiving prophylactic treatment.

The term “functional coat” means a coating on a drug particle which provides a controlled release of the drug (e.g., a sustained release), a delayed release of the drug (e.g., via an enteric coating), taste masking, salivary stimulation, a moisture barrier, texture modification, minimization of surface asperities, chip resistance, pliability or any combination of any of the foregoing.

In certain embodiments, the particulates are defined fuctionally with respect to the fact that they are of a size such that an effective dose cannot be delivered into the lower lung of a human patient. However, this definition should be understood to mean that a small percentage of drug (but not an amount effective to render a therapeutic effect) may in fact be inadvertently delivered to the lungs of the patient. Also, this definition is meant to define the particles, but not to limit the use of the invention to the treatments of humans only. The invention may be used for delivering doses of drugs to other mammals as well.

### BRIEF DESCRIPTION OF THE DRAWINGS

FIG. 1 is a graph of adhesion vs. humidity for standard powders.

FIG. 2 is a graph of adhesion vs. humidity for powders of the present invention.

FIG. 3 is a dissolution profile of Indomethacin & 4% PVP K-30 wet granulation in a pH 6.8 phosphate buffer made in accordance with an embodiment of the present invention.

FIG. 4 is a pH 6.8 phosphate buffer dissolution profile of Indomethacin & 10% PEG6000 melt granulation made in accordance with an embodiment of the present invention.

FIG. 5 is a 0.1 N Hydrochloric Acid dissolution profile of Indomethacin & 10% PEG6000 & 15% Acryl-eze melt granulation made in accordance with an embodiment of the present invention.

FIG. 6 is a pH 6.8 phosphate buffer dissolution profile of Indomethacin & 10% PEG6000 & 15% Acryl-eze melt granulation made in accordance with an embodiment of the present invention.

FIG. 7 is a 0.1 N Hydrochloric Acid dissolution profile of Indomethacin & 15% Sureteric & 10% PEG6000 melt granulation made in accordance with an embodiment of the present invention.

FIG. 8 is a 6.8 pH phosphate buffer dissolution profile of Indomethacin & 15% Sureteric & 10% PEG6000 melt granulation made in accordance with an embodiment of the present invention.

FIG. 9 is a 0.1 N Hydrochloric Acid dissolution profile of Indomethacin & 15% Sureteric melt granulation made in accordance with an embodiment of the present invention.

FIG. 10 is a 6.8 pH phosphate buffer dissolution profile of Indomethacin & 15% Sureteric melt granulation made in accordance with an embodiment of the present invention.

FIG. 11 is a 0.1 N Hydrochloric Acid dissolution profile of Indomethacin & 15% Sureteric & 10% Lustre Clear melt granulation made in accordance with an embodiment of the present invention.

FIG. 12 is a 6.8 pH phosphate buffer dissolution profile of Indomethacin & 15% Sureteric & 10% Lustre Clear melt granulation made in accordance with an embodiment of the present invention.

FIG. 13 depicts the particle size distribution for the formulations made in accordance with an embodiment of the present invention.

### DETAILED DESCRIPTION

In general, it has been recognized in the art that dry powder inhalation or insufflation formulations must consist of particles of a size of about 2 microns in diameter in order for the particles, when inhaled, to reach the peripheral or “deep” lung, including alveoli. Particles larger than 10 microns in diameter are not able to reach the deep lung when inhaled because they are collected on the back of the throat and upper airways in humans. Therefore, known powder delivery systems have been formulated with particle sizes of less than 10 microns in order for the particles to reach the intended site of action, the pulmonary system. Known powder delivery devices have not contemplated delivery of particles from a multi-dose delivery device to achieve gastrointestinal deposition, and therefore have avoided the use of drug particles having a large size, e.g. greater than 10 microns. By virtue of the invention disclosed in Applicants copending application, PCT/IB01/00251, it has been a surprising discovery that drug particles greater than 10 microns can be delivered from a multi-use drug delivery device for gastrointestinal deposition in a patient in order to minimize the inhalation of the drug particles into the lungs, in order to have substantially all of the dose deposited in the gastrointestinal system. By virtue of the present invention, it has been surprisingly discovered that powders that can be used in such devices can be functionally coated in order to provide desired characteristics with respect to their use in the device, e.g., increased flowability and decreased bridging (disclosed in more detail below) as well as characteristics of the powder itself, e.g. an acceptable weight variability. The powders can be used in the device or can be administered without the use of the device, e.g., by using a sachet.

In preferred embodiments, the drug formulation for gastrointestinal deposition of the invention comprising a non-compressed free flowing plurality of particles comprising a core comprising a drug and a pharmaceutically acceptable excipient, with the core overcoated with a functional coating.

In preferred embodiments, the core of the invention comprises drug coated with the excipient and a functional coat overcoating the excipient coat, thus providing a dual coated powder. The dual coated powder has improved functionality as a multiparticulate dosage form.

In other preferred embodiments, the core of the invention comprises drug interdispersed with the excipient and a functional coat overcoating the core. In these embodiments, the core can be prepared by wet granulation or by melt granulation. It has been surprisingly found that preparing the core by wet granulation or melt granulation results in a decreased fraction of fine particles in the resultant dosage form.

Depending on the choice of the initial excipient overcoat, single coated particles can have a surface area which is not smooth, with a significant degree of rugosity and surface asperities. Such particles have significant associated problems which decrease the usefulness and benefits of multiparticulate dosage forms.

For example, the presence of surface asperities on the surface of the particles provides gaps and cavernous areas which promote the coalescence of water onto the surface of the particles. The accumulation of water onto the surface of the particles promoted cohesiveness of the particles which is undesirous in the multiparticulate dosage form of the present invention, e.g., due to decreased flowability. Accordingly, the use of the present invention may not be able to be used to full benefit in areas which have increased humidity. This is relevant not only by the geographic location of use, e.g., a tropical area, but also relevant by the workplace, e.g. air conditioned buildings which may result in increased humidity. The functional overcoat can be provided in order to provide a relatively smooth surface area with minimal rugosity and surface asperities. The overcoated particles can then be resistant to the deleterious effects of moisture and humidity of the functionality of the multiparticulate dosage form. The moisture resistant overcoat may have the added benefit of protecting the stability of the drug contained therein.

Another functional problem associated with particles with increased rugosity and surface asperities is the presence of points or protrusions which rise from the surface of the particle and increase cohesiveness by multiple pathways.

One reason for increased adhesion between particles due to surface points or protrusions is due to physical interlocking between adjacent particles in the formulation. The protrusions of one particles can interlock between a “valley” in another particle. Alternatively, protrusions can actually interlock due to “jigsaw” type characteristics of the protrusions. The resultant is agglomeration of particles and decreased flowability of the formulation. An overcoat which smooths the surface can minimize asperities and rugosity and increase the functionality of the formulation.

Another reason for increased adhesion between particles due to surface points or protrusions is due to the fact that charge tends to gather at these points and protrusions. Thus, the existence of localized charge can increase electrostatic forces between the particles and promote agglomeration and adhesion. An overcoat which smooths the surface of the underlying particle and decreases asperities and rugosity can decrease accumulation and adhesion due to electrostatic forces. Electrostatic forces can also be minimized by coating a substrate with a conductive polymer, disclosed in more detail below.

The concept of rugosity of particles can be quantified by a rugosity index. The calculation of the rugosity index involves the concept of a “convex hull”. A convex hull is a minimum enveloping boundary fitted to an outline of the measured particle that is nowhere concave. The rugosity index is defined as the perimeter of the particles outline divided by the perimeter of the convex hull. According to this index, certain embodiments of the multiparticulates of the present invention can have a mean rugosity index of between 1.0 and 1.5, more preferably from about 1.0 to about 1.2. In other embodiments, greater than 80% of the particles of the invention have a rugosity index within the disclosed mean range. In other embodiments, greater than 90% of the particles of the invention have a rugosity index within the disclosed mean range.

Another calculation index which can be used in the present invention is a roundness index. When the particles of the present invention are coated as disclosed herein, certain embodiments will exhibit a roundness of the particles. The roundness index can be calculated as the square of the perimeter of the particles outline divided by 4π (cross-sectional or projection area of particle outline). According to this index, certain embodiments of the multiparticulates of the present invention can have a mean roundness index of between 0.70 and 1.0, more preferably from about 0.85 to about 1.0. In other embodiments, greater than 80% of the particles of the invention have a roundness index within the disclosed mean range. In other embodiments, greater than 90% of the particles of the invention have a roundness index within the disclosed mean range.

In certain embodiments of the invention, flowability is improved by virtue of the functional coatings, without the need for certain flow aids known in the art such as the inclusion of silicone dioxide. The use of silicone dioxide is not preferred in the present invention because this compound is not suited for inhalation, should a patient accidentally or inadvertently have aspiration into the lungs of a fraction of the unit dose.

Adhesion and agglomeration also leads to the concept of bridging which is particularly problematic with respect to the use of the multiparticulate formulation disclosed herein in multiple unit dosing devices. When multiple unit doses of the multiparticulates of the present invention are stored in containers, e.g., reservoirs, and unloaded therefrom through an opening or openings in the bottom of the container, the containers are often designed to have very steep walls adjacent the opening to aid the outward flow of the multiparticulates. Nevertheless the multiparticulates can become clogged and will have reduced or no flow out of the container. This phenomenon is generally termed “bridging” since the bulk material tends to assume a curved or cupola-like shape. It is known that sometimes vibrating or knocking the container walls from outside is sufficient to break the integrity of the bridge enable the flow to return to normal. Sometimes, however, such vibrating or knocking results in container wall vibrations which further compact the material resulting in an even more rigid and indestructible bridge being formed, or the shaking and vibrating of the container can break or damage the dosing device.

One aspect of the present invention is formulating the mean particles size of the particulates to have a diameter which can minimize or possibly eliminate bridging when the formulation is included in a system in a multiple unit dosing device (e.g., a hopper base device). The multiple unit dosing devices as disclosed herein and in PCT/IB01/00251 may be susceptible to bridging which could result in reduced flow and inaccurate dosing. It has been discovered that bridging can be significantly reduced if the particles size of the multiparticulates are no greater than 1/14th or 1/15th the diameter of the exit opening in the reservoir or container of the bulk formulation. The typical opening of a multiple unit dosing device is about 7 mm, thus, a preferred particle size of the present invention is a mean particles size of less than about 500 micrometers. If the mean particle size of the multiparticulates are significantly greater than 1/14th the size of the diameter of the exit opening, the resultant bridging and reduced flow will increase. For example, bridging may be more problematic if the mean particle size of the formulation is 1.5 mm in a dosing device with a 7 mm exit. Bridging is also increased if the particulates have asperities and protrusions due to interlocking as discussed above. With interlocking, the particles cannot move relative to each other in the direction of an applied driving force component, such as gravity, due to the presence of a force such as a frictional force component which is larger than the driving force component and normal thereto and which urges the particles against each other. The frictional force component that holds the particles together is proportional to the coefficient of friction of the particular bulk material. Thus, materials having relatively large coefficients of friction have a relatively large tendency to bridge. The inclusion of a coating or overcoating which smooths the surface of the multiparticulates will result in decreased bridging due to decreased interlocking.

The multiparticulates of the present formulation, when in motion are known to have a relatively smaller coefficient of friction than at rest. The present invention is therefore directed to devices which reduce the coefficient of friction between multiparticulates by producing relative motion therebetween in order to reduce bridging effects. This can be accomplished, for example, by the inclusion of a internal rake or lever which agitates and moves the particles within the device upon actuation, or by a vibrating mechanism which is preferably activated upon actuation.

The present invention is therefore directed to particles having a novel size range, which are dependent on a number of factors. In order to reduce pulmonary inhalation, the mean diameter of the particles are preferably greater than about 10 micrometers and preferably greater than about 50 micrometers and the mean diameter of the multiparticulates are preferably less than about 500 micrometers as a typical dosing device will have an exit opening of about 7 mm. However, this range is not meant to be limiting as the dosing devices (e.g., hopper base devices) can have different size openings and the formulations of the present invention may be used without the device.

As bridging and aspiration will depend on the actual size of the particles in proximity to each other, mean particles size is only one factor to consider, as the actual particles in proximity to each other may wind up being very large or very small, despite the mean particles size of the entire batch.

Accordingly, with respect to aspiration, it is preferred that greater than 90% of said particles have a diameter of greater than about 10 μm. Preferably, greater than 95% of said particles have a diameter of great than about 10 μm. More preferably, greater than 99% of said particles have a diameter of greater than about 10 μm.

In other embodiments, greater than 90% of said particles have a diameter of greater than about 50 μm. Preferably, greater than 95% of said particles have a diameter of great than about 50 μm. More preferably, greater than 99% of said particles have a diameter of greater than about 50 μm.

In other embodiments, greater than 90% of said particles have a diameter of less than about 500 μm. Preferably, greater than 95% of said particles have a diameter of less than about 500 μm. More preferably, greater than 99% of said particles have a diameter of greater than about 500 μm.

In other embodiments, greater than 90% of said particles have a diameter of greater than about 50 μm and greater than 90% of said particles have a diameter of less than about 500 μm. Preferably, greater than 95% of said particles have a diameter of great than about 50 μm and greater than 95% of said particles have a diameter of less than about 500 μm. More preferably, greater than 99% of said particles have a diameter of greater than about 50 μm and greater than 99% of said particles have a diameter of greater than about 500 μm.

In order to achieve the desired lower limit of the particles size of the present invention the invention, in certain embodiments is directed to a method of preparation comprising air jet sieving particles to remove fine particles. In particular embodiments, the invention is directed to a method of preparing a multiparticulate drug formulation for gastrointestinal deposition comprising preparing a non-compressed free flowing plurality of particles comprising a core comprising a drug and a pharmaceutically acceptable excipient as disclosed herein and air jet sieving the particles to separate the cores from fine particles; and thereafter overcoating said core with a functional coating as disclosed herein. The invention is also directed to compositions obtained using these methods.

The compositions of multiparticulates obtained using air jet sieving and methods thereof are not limited to the particular embodiments disclosed herein. Air jet sieving can be used for any composition of multiparticulates intended for oral use in order to remove fine particles (e.g., particles which may be aspirated into the lungs). Accordingly, the present invention is directed to compositions and methods of preparing a multiparticulate formulations for oral delivery comprising preparing a multiparticulate composition and air jet sieving the composition to remove particles of less than about 10 μm, less than about 50 μm or less than about 100 μm. In preferred embodiments, particles larger than about 500 μm or larger than about 1 mm are also removed from the composition. Preferably, multiple unit doses of the composition are then placed in an oral delivery device capable of metering a unit dose of the composition for oral delivery. These compositions can be coated (e.g. for sustained release or tastemasking) before air jet sieving, after air jet sieving or not coated at all. The coated embodiments can be single or multiple coated (e.g., as disclosed herein).

The use of an air jet sieve is beneficial as the standard sieving techniques used with screens and meshes may not separate all of the desired fine particles as the fine particles may adhere to the surface of larger particles and thus not separate during the sieving process. The air jet sieving process utilizes a negative pressure to draw particles below a particular size range down through an appropriate screen or mesh. In another embodiment, there is a combination of a downward negative pressure and an upward positive pressure which facilitates the de-agglomeration of the different particle sizes. In other embodiments, the upward pressure can be introduced upwards from a rotating wand. An apparatus utilizing a negative downward pressure and an upward positive pressure through a rotating wand is a Micron Air Jet Sieve MAJS I/II manufactured by Hosakawa.

In order to facilitate swallowing of a unit dose of the present formulation, excipient should be kept to a minimum in order to reduce the mass of the dose. Therefore, in preferred embodiments of the present invention, the drug particles comprise at least about 40% drug, at least about 50% drug, at least about 60% drug, at least about 80% drug, or at least about 90% drug.

In preferred embodiments, the core comprises drug coated with excipient; drug interdispersed in excipient; a combination thereof or drug coated onto excipient, e.g., drug coated inert beads. The core of drug and excipient is then overcoated with a functional coating. This is not limiting however, as it is contemplated that single coated particles and cores containing only drug (with at least one coating) are contemplated by the invention, as long as the desired functional characteristics are met. In preferred embodiments, the core is formed by mixing drug with excipient (e.g. a binder such as polyvinylpyrrolidone) to form a granulate which is then sieved and coated with further excipient (e.g. ethylcellulose). These cores can then be coated with a functional coating (e.g. microcrystalline cellulose).

In certain embodiments, wet granulation techniques can be used to prepare cores with the drug interdispersed in excipient. Utilizing wet granulation in preparing the core reduces any resultant fine particles in the final formulation. Reducing the fine particles results in an oral formulation which has decreased potential for pulmonary deposition due to the presence of respirable fine particles. The application of the functional coat of the invention results in a further decrease in respirable fine particles.

In certain embodiments, melt granulation techniques can be used to prepare the cores with the drug interdispersed in excipient. In certain embodiments, melt granulation of the drug with excipient results in a smaller fraction of respirable fine materials as compared to wet granulation techniques. In certain embodiments, in order to provide an equivalent reduction of respirable fines with wet granulation techniques as compared to melt granulation techniques, it is necessary to increase the amount of functional coat. An increase in functional coat can result in a delayed drug release with variable batch to batch dissolution rates. In certain embodiments, final products prepared with a melt granulation step has minimal batch to batch variability and an acceptable drug release profile, e.g., without an unwanted delay. As with wet granulation embodiments, the application of the functional coat of the invention results in a further decrease in respirable fine particles.

In certain embodiments, melt granulation can be used in preparing the core in addition to wet granulation. For example, a fine material with a large surface area would require an increased amount of melt granulation excipient. In such embodiments, the fine particles can be wet granulated in order to provide large particles with a decreased surface area, while at the same time, reducing respirable particles. The resultant wet granulated particles can then be melt granulated with a suitable excipient, which can result in a further reduction of respirable particles.

In certain embodiments, melt granulation can be used prior to, or after the application of the functional coat. For example, if the functional coat is an enteric coating, the melt granulation can be performed before application of the enteric coat, or enteric coated drug particles can be melt granulated with the melt granulation excipient. Both alternatives would result in a reduction of respirable particles as compared to the formulations without the melt granulation before or after the application of the enteric coat. In certain embodiments, performing the melt granulation prior to application of the functional coat results in a less variable batch to batch ratio as compared to performing the melt granulation after the application of the functional coat. In certain embodiments, performing the melt granulation prior to the application of the functional coat results in a more acceptable particle size distribution for applying the functional coat, due to the increased reduction of fine particles.

When applying the functional coat, e.g., an enteric coat to the melt granulated core, it is preferable to have a difference between the melting point of the melt granulation excipient and the film forming temperature of the coating agent of 20 degrees C. or more, in order to reduce interdispersion of the melt granulated material and the functional coat.

Suitable melt granulations excipients for the present invention include, e.g., wax materials such as beeswax, white wax, emulsifying wax, hydrogenated vegetable oil, cetyl alcohol, stearyl alcohol, free wax acids such as stearic acid; esters of wax acids; propylene glycol monostearate, glyceryl monostearate; and carnauba wax. The wax material can be a water insoluble wax material or a non-polymeric wax material. In certain preferred embodiments, the melt granulation excipient is glyceryl monostearate, a glyceryl stearate, glyceryl palmitostearate, glyceryl behenate, stearyl alcohol, stearic acid, or a combination thereof.

Other suitable melt granulation excipients include polyethylene glycols which can have a weight average molecular weight of from about 100 to about 10,000, from about 200 to about 1000, or from about 200 to about 400. Preferably, the polyethylene gycol has a molecular weight of from about 4,000 to about 8,000 and most preferably a molecular weight of about 6,000.

In certain embodiments, the melt granulation is transferred to a tray for cooling, rather than cooling the granulation while mixing as cooling the granulation while mixing may result in fragmentation of the granules. Such fragmentation can result in an increased percentage of unwanted respirable fines.

In certain embodiments, the excipient of the core provides a controlled release (e.g., a sustained release) of the drug upon gastrointestinal deposition. For example, the excipient can provide a controlled release of the drug upon gastrointestinal deposition to provide a therapeutic effect for at least 12 hours after oral administration. In other embodiments, the excipient can provide a controlled release of the drug upon gastrointestinal deposition to provide a therapeutic effect for at least 24 hours after oral administration.

In other embodiments, the excipient can provide a delayed release (e.g., via an enteric coating) of the drug upon gastrointestinal deposition, such as delaying release of the drug to effect intestinal absorption for drugs irritating to the gastric mucosa.

In other embodiments, the excipient can provide tastemasking. This is especially beneficial for bitter tasting drugs, especially when administered to small children. If a dose of drug intended for a child has a bad taste, the child may spit out the dose resulting in waste and a possible reduction in the amount administered. An overdose is also possible as if the dose is administered again, it is possible that the child already ingested a portion of the previous dose.

In other embodiments, the excipient can include a salivary stimulant to promote the production of saliva to facilitate the swallowing of the unit dose. This is especially useful in patients with xerostomia.

In other embodiments, the excipient can provide a moisture barrier in order to reduce the coalescence of water on the surface of the particles and reduce undesirable cohesiveness over a wide range of humidities. In certain embodiments, the cohesiveness of the particles does not substantially change over a humidity gradient from about 20% relative humidity to about 80% relative humidity. In other embodiments, the cohesiveness of the particles does not substantially change over a humidity gradient from about 40% relative humidity to about 60% relative humidity.

The effect of humidity can have a negative impact of the flowability of particles (e.g., due to cohesiveness). Flowability of the particles can be measured by such tests as the Carr consolidation index, the uniaxial compression test and the Jenike shear test. The tests can be performed over a range of relative humidities in order to evaluate the moisture resistance of the present invention.

The Carr consolidation index is measured as Tapped Density−Bulk Density×100

Tapped Density

The relation between Carr's index and powder flowability is expressed in the table below:

| Carr's Index | State of Flowability |
|---|---|
| 5-15 | Excellent |
| 12-16 | Good |
| 18-21 | Fair |
| 23-35 | Poor |
| 33-38 | Very Poor |
| >40 | Very, Very Poor |

In certain embodiments of the invention, the flowability according to Carr's index over a humidity gradient from about 20% relative humidity to about 80% relative humidity is preferably 21 or less, preferably 16 or less and most preferably 12 or less. In other embodiments, the Carr's index does not change by more than about 20%, preferably does not change by more than 10%, most preferably does not change by more than 5%, over a humidity from about 20% relative humidity to about 80%. In other embodiments, the composition has the above characteristics over a humidity gradient from about 40% relative humidity to about 60% relative humidity or 10% to about 90% relative humidity.

In the uniaxial compression test, a hollow split cylinder is filled with the test powder. A force transducer is used to apply force or a weight from the top of the cylinder onto the powder to consolidate it in a vertical direction for a short known time. The applied consolidation force (σ₁) is then recorded. Then the hollow split cylinder is removed from around the consolidated powder. Thereafter increasing vertical load is applied onto the powder until the consolidated powder collapses or crackers. This new weight force (σ_{c}) is noted. The smaller this value is the better the flowability of the powder. The value (ffc) usually known as the quotient of consolidation stress and the unconfined yield strength is then calculated by σ₁ divided by σ_{c}

The larger this value, the better the flowability of the powder. If the value is >10, the powder is free flowing. If it is between 4-10, the powder shows adequate flow.

In certain embodiments of the invention, the flowability according to the uniaxial compression test over a humidity gradient from about 20% relative humidity to about 80% relative humidity is preferably greater than about 4, preferably greater than about 10 and most preferably greater than about 12. In other embodiments, the uniaxial compression test does not change by more than about 20%, preferably does not change by more than 10%, most preferably does not change by more than 5%, over a humidity from about 20% relative humidity to about 80%, more preferably. In other embodiments, the composition has the above characteristics over a humidity gradient from about 40% relative humidity to about 60% relative humidity or 10% to about 90% relative humidity.

The Jenike shear test involves the use of a cell consisting of a base, a ring that rests on the base, a mold ring, a preconsolidation lid and shearing lid. The cell is first filled with the test powder using a spoon. The preconsolidation lid is then placed on the powder and a pre-shear stress is applied on it. The sample is then consolidated by applying a number of 90° twists to the lid. A horizontal shearing force is then applied to the ring at a rate of 2 mm per minute until the consolidated powder collapses. The ffc can then be calculated as above. Preferably, the flowability of the powder over a humidity range according to the Jenike shear test is the same as with respect to the uniaxial test as disclosed above.

In other embodiments, the excipient provides a texture modifier in order to improve mouthfeel of the unit dose in the mouth. An increase in palatability would be expected to increase compliance as patients may be unwilling to take multiple or chronic dosing of a formulation which they perceived to be objectionable.

In other embodiments, the functional coating can have the same affect as disclosed above with respect to the excipient coating.

For example, the functional coating can provide a controlled or delayed release of the drug upon gastrointestinal deposition; the functional coating can provide tastemasking; the functional coating can comprise a salivary stimulant; the functional coating can provide a moisture barrier; or the functional coating can be a texture modifier. The present invention is contemplated to encompass all combinations of functional coating with particular characteristics of core excipient. It is also understood that one or more of the functions and characteristics of the excipient and overcoating can be achieved with a single coating. For example, an overcoat which provides a moisture barrier, may also provide texture modification. The same is true in the core, for example, when the core is coated with an excipient that provides controlled release and tastemasking of the underlying drug.

In a preferred embodiment, the functional coating minimizes asperities on the surface of the particles to provide the beneficial characteristics disclosed above, e.g. reduced static and reduced interlocking.

The desired flow characteristics and reduced adhesion and agglomeration of the multiparticulates of the present invention are better achieved when the coating or coatings of the particles have pliability and are not brittle, with a resistant to chipping. Brittleness can increase surface asperities and reduce the smoothness of the outer coating. Further, chipping can result in the presence of small particles which can aspirated into the lungs. Thus, it is desirous to have a pliable tough film which is deformable (pliable) and resistant to chipping (tough).

The pliable tough film of the present invention can be achieve by the manipulation of the process and materials of the coating. In certain embodiments, a plasticizer can be used in the functional coating in order to make the particles pliable.

Also, the desired pliable tough film can be obtained by minimally including or not including ingredients which can promote brittleness of the coating. In certain embodiments of the invention, the use of lakes and opacifiers are minimally used or not used at all as the increased use of such ingredients can promote brittleness. In certain embodiments, a colorant which is not a lake or an opacifier can be used and the lake or opacifier is not used at all in order to maintain the integrity of the coating. Other embodiments are directed to including plasticizer and coloring agents in a ratio which results in a coating having a desired pliability and non-brittleness.

In certain preferred embodiments of the invention, the multiparticulate dosage form has minimal adhesion and non-agglomeration over a broad range of humidity. A low humidity dry environment tends to promote adhesion and agglomeration of particles due to electrostatic forces. The functional coating of the present invention can provide a smooth surface to the particles in order to reduce the accumulation of charge in protrusions and to keep the dosage form from having increased particle to particle interaction.

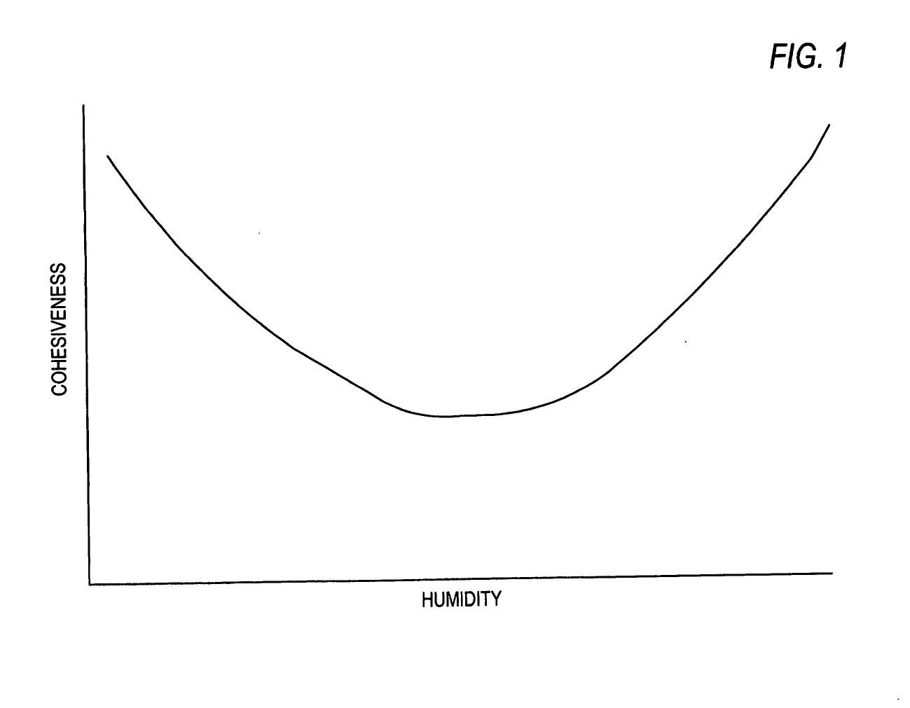

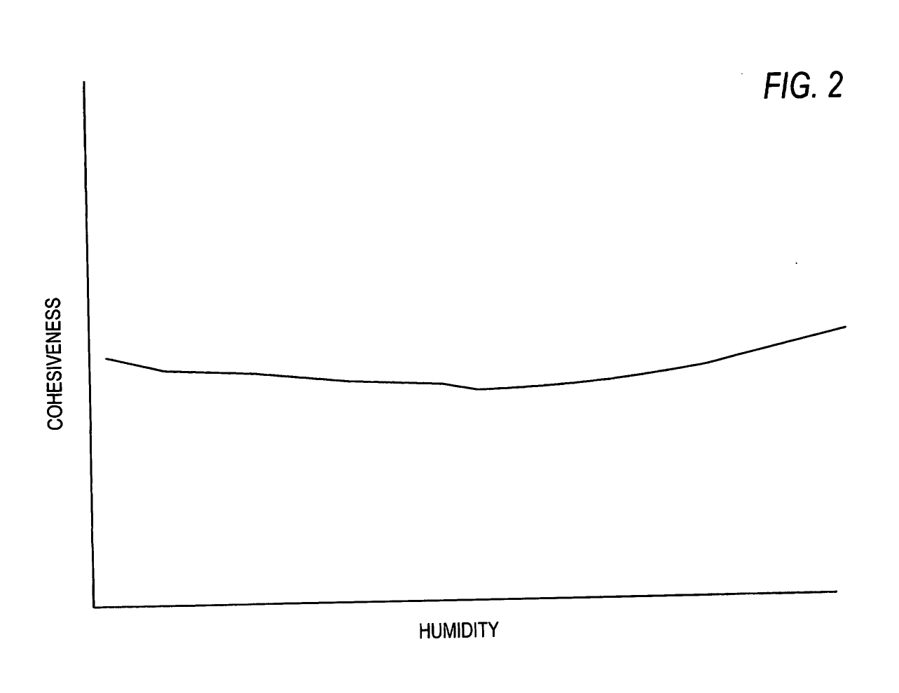

Likewise, an environment of increased humidity can promote adhesion of particles due to surface tension of water accumulating of the surface of the particles. The functional coating of the present invention can also provide a surface to the particles in order to reduce the coalescence of water on the surface and thus reducing surface tension and particle to particle interaction. This concept of decreased coalescence of water can be in addition to, or separate from the embodiment which reduces the accumulation of charge on the particles. FIG. 1 is a representative graph of typical powders plotting stickiness versus humidity. FIG. 2 is a representative graph of particles of the present invention, graphing stickiness versus humidity.

As previously discussed, the functional coating and the core excipient can provide overlapping characteristics. The following representative materials are meant to be used (i) in the functional overcoat of the core; (ii) the core excipient coat over the drug; (iii) interdispersed with then drug or (iv) any combination of (i), (ii) and (iii).

Controlled release materials useful in the present invention are preferably hydrophobic materials. The hydrophobic materials can be selected from the group consisting of an acrylic polymer, a cellulosic material, shellac, zein and mixtures thereof.

Preferably the hydrophobic material is an acrylic polymer. The acrylic polymer can be, e.g., selected from, the group consisting of acrylic acid and methacrylic acid copolymers, methacrylic acid copolymers, methyl methacrylate copolymers, ethoxyethyl methacrylates, cynaoethyl methacrylate, methyl methacrylate, copolymers, methacrylic acid copolymers, methyl methacrylate copolymers, methyl methacrylate copolymers, methyl methacrylate copolymers, methacrylic acid copolymer, aminoalkyl methacrylate copolymer, methacrylic acid copolymers, methyl methacrylate copolymers, poly(acrylic acid), poly(methacrylic acid, methacrylic acid alkylamide copolymer, poly(methyl methacrylate), poly(methacrylic acid) (anhydride), methyl methacrylate, polymethacrylate, methyl methacrylate copolymer, poly(methyl methacrylate), poly(methyl methacrylate) copolymer, polyacrylamide, aminoalkyl methacrylate copolymer, poly(methacrylic acid anhydride), glycidyl methacrylate copolymers and mixtures thereof.

When the controlled release material is a cellulosic material, the cellulosic material is, e.g., selected from the group consisting of cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, cellulose acetate butyrate and mixtures thereof.

Particularly preferred controlled release materials are ethylcellulose, polymethacrylates, e.g. Eudragit RL and RS, glyceryl behenate, methylcellulose and sodium carboxymethylcellulose.

In other embodiments of the invention, the controlled release material comprises a lacquer material. The lacquer material can be selected, e.g., from the group consisting of corn oil, cottonseed oil, menhaden oil, pine oil, peanut oil, safflower oil, sesame oil, soybean oil, linseed oil and mixtures thereof. Other suitable oils useful as lacquer materials include fatty acids of C8-C20 oils which can be saturated, unsaturated, glycerides thereof, and combination thereof. Preferably a salt such as magnesium stearate is included. Other suitable oils useful as lacquer materials include branched or polycarboxylated oils such as linoleic acid, linolenic acid, oleic acid and combinations thereof. Saturated oils from the following table are also useful as lacquer agents:

| Systematic | Trivial | Shorthand | Molecular | Melting point |
|---|---|---|---|---|
| name | name | designation | wt. | (° C.) |
| Octanoic | Caprylic | 8:0 | 144.2 | 16.7 |
| Decanoic | Capric | 10:0 | 172.3 | 31.6 |
| Dodecanoic | Lauric | 12:0 | 200.3 | 44.2 |
| Tetradecanoic | Myristic | 14:0 | 228.4 | 53.9 |
| Hexadecanoic | Palmitic | 16:0 | 256.4 | 63.1 |
| Heptadecanoic | Margaric | 17:0 | 270.4 | 61.3 |
| Octadecanoic | Stearic | 18:0 | 284.4 | 69.6 |
| Eicosanoic | Arachidic | 20:0 | 412.5 | 75.3 |
| Docosanoic | Behenic | 22:0 | 340.5 | 79.9 |
| Tetracosanoic | Lignoceric | 24:0 | 368.6 | 84.2 |

The use of lacquer agents may not release the drug of the multiparticulates. Therefore it may be necessary to include a channeling agent in an amount sufficient to provide the desired release of the drug, e.g., over 12 or 24 hours. Suitable channeling agents include polyvinylpyrrolidone, polyethyleneglycols, dextrose, sucrose, mannitol, xylitol and lactose. Antioxidants can also be added in order to reduce polymerization which leads to increased hardness.

The use of lacquer agents is beneficial as it reduces the amount of excipient needed to provide a controlled release of the drug from the particles of the present invention. In certain embodiments, less than about 1% lacquer is needed in the formulation (w/w) to provide the desired effect. Accordingly, as only a small amount of lacquer material is needed, it is preferably mixed with a dispersing agent. Suitable dispersing agents include colloidal silicone dioxide, talc, kaolin, silicone dioxide, colloidal calcium carbonate, bentonite, Fuller's earth, magnesium aluminum silicate and mixtures thereof. A preferred lacquer material is linseed oil with kaolin as a dispersing agent.

The lacquer material can be granulated with the drug in order to provide controlled release matrices or can coat the drug particulates. The use of lacquer materials is disclosed as providing controlled release in multiparticulate dosage forms. However, it also contemplated by the present invention that the use of lacquer agents with optional channeling agents and dispersing agents can also be used in solid dosage forms such as tablets. For example, an immediate release tablet core can be coated with sustained release coating comprising a lacquer agent as disclosed above with an optional channeling agent and dispersing agent. In these embodiments as well, a preferred lacquer material is linseed oil with kaolin as a dispersing agent.

Preferably, the delayed release material used in the present invention are enteric polymers. The enteric polymers can be selected from, e.g., the group consisting of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethyl-cellulose and mixtures thereof. Particularly preferred enteric polymers are polymethacrylates such as Eudragit US polymers, cellulose acetate phthalate, polyvinyl acetate phthalate, bydroxypropyl-methylcellulose phthalate and shellac. Sureteric™ is an example of a polyvinyl acetate phthalate based entereic coating. Acryl-eze™ is an example of a methacrylic acid copolymer based enteric coating.

The tastemasking material of the present material can be selected from, e.g., the group consisting of water-soluble sweetening agents, water-soluble artificial sweeteners, dipeptide based sweeteners and mixtures thereof. The water-soluble sweetening agent can be selected from, e.g., the group consisting of monosaccharides, disaccharides and polysaccharides such as xylose, ribose, glucose, mannose, galactose, fructose, dextrose, sucrose, sugar, maltose, partially hydrolyzed starch, or corn syrup solids and sugar alcohols such as sorbitol, xylitol, or mannitol and mixtures thereof. The water-soluble artificial sweetener material of the present invention is selected from, e.g., the group consisting of soluble saccharin salts, such as sodium or calcium saccharin salts, cyclamate salts, acesulfam-K, the free acid form of saccharin and mixtures thereof. The dipeptide based sweetener is preferably L-aspartyl L-phenylalanine methyl ester. Particularly preferred taste masking agents are glyceryl behenate, glyceryl palmitostearate, ethylcellulose and polymethacrylates such as Eudragit E, EPO and RD.

In other embodiments of the invention, the multiparticulates can comprise an effervescent compound or composition which provides a pleasing organoleptic effect which can substantially mask the taste of unpalatable active ingredients in the powder. The effervescent action also acts as a stimulant to saliva production. Effervescent agents include compounds which evolve gas. The preferred effervescent agents evolve gas by means of chemical reactions which take place upon exposure to a liquid such as saliva in the mouth. This bubble or gas generating chemical reaction is most often the result of the reaction of an acid (e.g. the saliva stimulant acids listed above) and an alkali metal carbonate/dicarbonate or base. The reaction of these two general classes of compounds produces carbon dioxide gas upon contact with saliva.

Other salivary stimulant of the present invention can be selected from, e.g., food acids, acid anhydrides and acid salts. Food acids include tartaric acid, malic acid, fumaric acid, adipic acid, and succinic acids and fruit acids, e.g., citric acid. Acid anhydrides of the above described acids may also be used. Acid salts may include sodium, dihydrogen phosphate, disodium dihydrogen pyrophosphate, acid citrate salts and sodium acid sulfite.

The moisture barrier material of the present invention can be, e.g., selected from the group consisting of acacia gum, acrylic acid polymers and copolymers (polyacrylamides, polyacryldextrans, polyalkyl cyanoacrylates, polymethyl methacrylates), agar-agar, agarose, albumin, alginic acid and alginates, carboxyvinyl polymers, cellulose derivatives such as cellulose acetate, polyamides (nylon 6-10, poly(adipyl-L-lysines, polyterephthalamides and poly-(terephthaloyl-L-lysines)), poly-.epsilon.-caprolactam, polydimethylsiloxane, polyesters, poly(ethylene-vinyl acetate), polyglycolic acid, polyactic acid and its copolymers, polyglutamic acid, polylysine, polystyrene, shellac, xanthan gum, anionic polymers of methacrylic acid and methacrylic acid esters, hydroxyalkylcelluloses and mixtures thereof. In certain embodiments, the moisture barrier material is a hydroxyalkylcellulose such as hydroxypropylmethylcellulose; a cellulosic material such as microcrystalline cellulose; carrageenan; or mixtures thereof. Particularly preferred moisture barrier materials are microcrystalline cellulose/carrageenan-based coating systems, such as LustreClear, ethylcellulose; such as Aquacoat ECD (formulated as a 50:50 mixture with hydroxypropylmethylcellulose) and polyvinyl alcohol based systems such as Opadry AMB. The above disclosed lacquer agents can also be used as moisture barriers.

The texture modifier material of the present invention can be, e.g., selected from the group consisting of acacia gum, acrylic acid polymers and copolymers (polyacrylamides, polyacryldextrans, polyalkyl cyanoacrylates, polymethyl methacrylates), agar-agar, agarose, albumin, alginic acid and alginates, carboxyvinyl polymers, cellulose derivatives such as cellulose acetate, polyamides (nylon 6-10, poly(adipyl-L-lysines, polyterephthalamides and poly-(terephthaloyl-L-lysines)), poly-epsilon.-caprolactam, polydimethylsiloxane, polyesters, poly(ethylene-vinyl acetate), polyglycolic acid, polyactic acid and its copolymers, polyglutamic acid, polylysine, polystyrene, shellac, xanthan gum, anionic polymers of methacrylic acid and methacrylic acid esters, hydroxyalkylcelluloses and mixtures thereof. Particularly preferred texture modifiers are cellulose, e.g., carboxymethyl cellulose and microcrystalline cellulose; polydextrose; modified starch; dextrins; gums, e.g. xanthan, guar, locust-bean, carrageenan and alginates; pectins; maltodexrins and carbomers.

Materials which can be used to obtain a pliable and/or chip resistant coating of the present invention can be selected, e.g., from the group consisting of acacia gum, alginic acid and alginates, carboxymethylcellulose, ethylcellulose, gelatine, hydroxypropylcellulose, hydroxypropylmethylcellulose, methylcellulose, xanthan gum, pectin, tragacanth, microcrystalline cellulose, hydroxyethylcellulose, ethylhydroxyethylcellulose, sodium carboxymethylcellulose, polyethylene glycols, polyvinylpyrrolidone, polyvinyl alcohol, polyacrylic acid, gum arabic, lactose, starch (wheat, maize, potato and rice starch), sucrose, glucose, mannitol, sorbitol, xylitol, stearic acid, hydrogenated cottonseed oil, hydrogenated castor oil, vinylpyrrolidone-vinyl acetate copolymers, fructose, methylhydroxyethylcellulose, agar-agar, carrageenan, karaya gum, chitosan, starch hydrolysates and mixtures thereof. Especially preferred materials are plasticizers which can be selected from, e.g., the group consisting of dibutyl sebacate, diethyl phthalate, triethyl citrate, tibutyl citrate, triacetin, benzyl benzoate, chlorobutanol, sorbitol, glycerol, polyethyleneglycol and mixtures thereof.

With respect to decreasing static in the particles, it was disclosed above that a smooth surface can be provided to the surface of the particles in order to avoid charge gathering and decrease adhesion and agglomeration of particles. Decreasing charge can also be effected on the particles of the present invention by including a conductive polymer into the functional coat. Examples of conductive polymers are polypyrroles, polythiophene, poly(p-phenylene), poly(phenylene vinylene) and trans-polyacetylene. These are rigid polymers and may require the addition of a plasticizer in order to provide a more flexible coating. A less rigid conductive polymer is polyanilene, although inclusion of a plasticizer is still preferable.

A preferred method to decrease charge on the multiparticulates is by the electrohydrodynamic spraying of a viscous and highly conductive polyvinyl alcohol aqueous solution, as described in Electrospraying of a highly conductive and viscous liquid, Speranza et al. Journal of Electrostatics, (51) p494, hereby incorporated by reference.

Conductive polymers are further discussed in U.S. Pat. Nos. 6,060,116 and 5,268,407, hereby incorporated by reference with respect to their combination with the multiparticulate formulations of the present invention.

Another method of reducing charge in the present invention is to include in the multiparticulates, or provide a final coat of compounds selected from magnesium stearate and the like, surfactants such as sodium lauryl sulphate and combinations thereof. In order for these materials to be most effective, they would be included as a final coat with robust mixing in order to provide an even coat on the particles.

Classes of drugs which are suitable in the present invention include antacids, anti-inflammatory substances, coronary dilators, cerebral dilators, peripheral vasodilators, anti-infectives, psychotropics, anti-manics, stimulants, anti-histamines, laxatives, decongestants, vitamins, gastro-intestinal sedatives, anti-diarrheal preparations, anti-anginal drugs, vasodilators, anti-arrhythmics, anti-hypertensive drugs, vasoconstrictors and migraine treatments, anti-coagulants and anti-thrombotic drugs, analgesics, anti-pyretics, hypnotics, sedatives, anti-emetics, anti-nauseants, anti-convulsants, neuromuscular drugs, hyper- and hypoglycemic agents, thyroid and anti-thyroid preparations, diuretics, anti-spasmodics, uterine relaxants, mineral and nutritional additives, anti-obesity drugs, anabolic drugs, erythropoietic drugs, anti-asthmatics, bronchodilators, expectorants, cough suppressants, mucolytics, drugs affecting calcification and bone turnover and anti-uricemic drugs.

Specific drugs include gastro-intestinal sedatives such as metoclopramide and propantheline bromide; antacids such as aluminum trisilicate, aluminum hydroxide, ranitidine and cimetidine; anti-inflammatory drugs such as phenylbutazone, indomethacin, naproxen, ibuprofen, flurbiprofen, diclofenac, dexamethasone, prednisone and prednisolone; coronary vasodilator drugs such as glyceryl trinitrate, isosorbide dinitrate and pentaerythritol tetranitrate; peripheral and cerebral vasodilators such as soloctidilum, vincamine, naftidrofuryl oxalate, co-dergocrine mesylate, cyclandelate, papaverine and nicotinic acid; anti-infective substances such as erythromycin stearate, cephalexin, nalidixic acid, tetracycline hydrochloride, ampicillin, flucloxacillin sodium, hexamine mandelate and hexamine hippurate; neuroleptic drugs such as flurazepam, diazepam, temazepam, amitryptyline, doxepin, lithium carbonate, lithium sulfate, chlorpromazine, thioridazine, trifluperazine, fluphenazine, piperothiazine, haloperidol, maprotiline hydrochloride, imipramine and desmethylimipramine; central nervous stimulants such as methylphenidate, ephedrine, epinephrine, isoproterenol, amphetamine sulfate and amphetamine hydrochloride; antihistamic drugs such as diphenhydramine, diphenylpyraline, chlorpheniramine and brompheniramine; anti-diarrheal drugs such as bisacodyl and magnesium hydroxide; the laxative drug, dioctyl sodium sulfosuccinate; nutritional supplements such as ascorbic acid, alpha tocopherol, thiamine and pyridoxine; anti-spasmodic drugs such as dicyclomine and diphenoxylate; drugs affecting the rhythm of the heart such as verapamil, nifedipine, diltiazem, procainamide, disopyramide, bretylium tosylate, quinidine sulfate and quinidine gluconate; drugs used in the treatment of hypertension such as propranolol hydrochloride, guanethidine monosulphate, methyldopa, oxprenolol hydrochloride, captopril and hydralazine; drugs used in the treatment of migraine such as ergotamine; drugs affecting coagulability of blood such as epsilon aminocaproic acid and protamine sulfate; analgesic drugs such as acetylsalicylic acid, acetaminophen, codeine phosphate, codeine sulfate, oxycodone, dihydrocodeine tartrate, oxycodeinone, morphine, heroin, nalbuphine, butorphanol tartrate, pentazocine hydrochloride, cyclazacine, pethidine, buprenorphine, scopolamine and mefenamic acid; anti-epileptic drugs such as phenyloin sodium and sodium valproate; neuromuscular drugs such as dantrolene sodium; substances used in the treatment of diabetes such as tolbutamide, disbenase glucagon and insulin; proteins and peptides such as heparin and calcitonin, drugs used in the treatment of thyroid gland dysfunction such as triiodothyronine, thyroxine and propylthiouracil, diuretic drugs such as furosemide, chlorthalidone, hydrochlorthiazide, spironolactone and triamterene; the uterine relaxant drug ritodrine; appetite suppressants such as fenfluramine hydrochloride, phentemmine and diethylproprion hydrochloride; anti-asthmatic and bronchodilator drugs such as aminophylline, theophylline, salbutamol, orciprenaline sulphate and terbutaline sulphate; expectorant drugs such as guaiphenesin; cough suppressants such as dextromethorphan and noscapine; mucolytic drugs such as carbocisteine; anti-septics such as cetylpyridinium chloride, tyrothricin and chlorhexidine; decongestant drugs such as phenylpropanolamine and pseudoephedrine; hypnotic drugs such as dichloralphenazone and nitrazepam; anti-nauseant drugs such as promethazine theoclate; haemopoietic drugs such as ferrous sulphate, folic acid and calcium gluconate; uricosuric drugs such as sulphinpyrazone, allopurinol and probenecid; and calcification affecting agents such as biphosphonates, e.g., etidronate, pamidronate, alendronate, residronate, teludronate, clodronate and alondronate.

Drugs which possess taste and/or odor characteristics which, when administered orally without any excipients, render the drug or therapeutic agent unpalatable to a subject and would be candidates for taste masking in the present invention include, but are not limited to, H₂ receptor antagonists, antibiotics, analgesics, cardiovascular agents, peptides or proteins, hormones, anti-migraine agents, anti-coagulant agents, anti-emetic agents, anti-hypertensive agents, narcotic antagonists, chelating agents, anti-anginal agents, chemotherapy agents, sedatives, anti-neoplastics, prostaglandins, antidiuretic agents and the like. Typical drugs include but are not limited to nizatidine, cimetidine, ranitidine, famotidine, roxatidine, etinidine, lupitidine, nifentidine, niperitone, sulfotidine, tuvatidine, zaltidine, erythomycin, penicillin, ampicillin, roxithromycin, clarithromycin, psylium, ciprofloxacin, theophylline, nifedipine, prednisone, prednisolone, ketoprofen, acetaminophen, ibuprofen, dexibuprofen lysinate, flurbiprofen, naproxen, codeine, morphine, sodium diclofenac, acetylsalicylic acid, caffeine, pseudoephedrine, phenylpropanolamine, diphenhydramine, chlorpheniramine, dextromethorphan, berberine, loperamide, mefenamic acid, flufenamic acid, astemizole, terfenadine, certirizine, phenyloin, guafenesin, N-acetylprocainamide HCl, pharmaceutically acceptable salts thereof and derivatives thereof.

Particularly preferred agents include antibiotics such as clarithromycin, amoxicillin erythromycin, ampicillin, penicillin, cephalosporins, e.g., cephalexin, pharmaceutically acceptable salts thereof and derivatives thereof.

Other preferred agents are acetaminophen and NSAIDS such as ibuprofen, indomethacin, aspirin, diclofenac and pharmaceutically acceptable salts thereof.

The size of the unit dose is dependent on the amount of drug needed to provide the intended therapeutic effect and the amount of any pharmaceutically acceptable excipient which may be necessary. Typically, a unit dose of from about 0.01 mg to about 1.5 g would be sufficient to contain a therapeutically effective amount of the drug to be delivered, however, this range is not limiting and can be smaller or higher, depending on the amount of drug and excipient that is necessary.

### DETAILED DESCRIPTION OF CERTAIN EMBODIMENTS

### EXAMPLE 1

### Controlled-Release Propranolol HCl

Step 1: Granulation of Propranolol HCl

Prior to commencing granulation of the Propranolol HCl, the vessel of the MP Micro is pre-warmed by heating at 70° C. for 15 minutes with a nominal airflow of 6.0 m³/Hr. 76 g of Propranolol HCl and 4 g of PVP K-30 is added to the vessel and the process temperature set to 70° C. The airflow is then increased until the product is fluidised. Once a constant temperature is achieved within the powder bed, spray granulation of the product is commenced by the introduction of distilled water as the granulation fluid. An atomising pressure of 2 bar is used.

Once the material is granulated the addition of the granulation fluid is stopped and the powder bulk is dried. The end point of the drying process is indicated by a constant temperature within the powder bed. At this point the temperature of the inlet air is reduced to 25° C. and the bulk material removed. Once cooled the material is screened through a 250 micron sieve then air jet sieved to remove particles below 100 microns.

Step 2: Spray Coating With Surelease

An aqueous dispersion of Surelease is prepared by diluting to 15% w/w solids (i.e. 60% Surelease dispersion and 40% distilled or deionised water) and stirred using a low shear mixer for approximately 15 minutes. With the precision coater module attached the vessel is preheated at 70° C. for 15 minutes with a nominal airflow of 6.0 m³¹ Hr. 60 g of the granulated Propranolol HCl is returned to the MP Micro and the process temperature set at 70° C. to achieve a product temperature of 40-45° C. The airflow is increased until the product is fluidised. Once the material is equilibrated within the vessel, a constant temperature is reached within the powder bed. The material is then sprayed with the Surelease dispersion to achieve a 10-30% wt. gain depending on the desired release profile at a spraying rate of 1.0 g/min with an atomising air pressure of 2 bar. Once the desired weight of Surelease coating are added to the granules the pump and the atomising air are stopped and the material dried until the powder bed reaches a constant temperature. At this point the inlet air temperature is reduced to 25° C. and the operation stopped.

Step 3: Overcoating with LustreClear

A 9% w/w dispersion of LustreClear is prepared as follows:
The necessary quantity of LustreClear film coating system is accurately weighed out.
The necessary quantity of water is accurately weighed into the mixing vessel.
With the propeller in the centre and as close to the bottom of the vessel as possible, the water is stirred to form a vortex without drawing air into the liquid.
The LustreClear powder is steadily added to the vortex, avoiding powder flotation on the liquid surface.
The stirrer speed is increased in order to maintain the vortex as required.
After all the LustreClear is added, the dispersion is then mixed for a further 3 hours.
The dispersion is then left for a further 2 hours before use.

Residual Surelease is removed from the spray nozzle by rapidly flushing through with the 9% w/w dispersion LustreClear. The precision coater module is washed and then dried by heating at 85° C. for 15 minutes with a nominal airflow of 6.0 m³/Hr. The Surelease coated granules are placed into the precision coater and are fluidised using the same conditions as for the addition of Surelease. Once the material is equilibrated within the vessel, a constant temperature is reached within the powder bed. At this point the granules are coated with the 9% w/w dispersion of LustreClear at a rate of 1.0 g/min. Once a coating of 4-30% wt. gain is applied, spraying of the LustreClear dispersion is stopped and the material dried until a constant temperature is observed within the powder bed. At this point the airflow temperature is reduced to 25° C. and the bulk material removed, allowing it to cool.

### EXAMPLE 2

### Enteric Coated Indomethacin

Step 1: Granulation of Indomethacin

Before commencing the granulation of the Indomethacin, the vessel of the MP Micro is pre-warmed by heating at 70° C. for 15 minutes with a nominal airflow of 6.0 m³/Hr. 76 g of Indomethacin and 4 g of PVP K-30 is added to the vessel and the process temperature set to 70° C. The airflow is then increased until the product is fluidised. Once a constant temperature is achieved within the powder bed, spray granulation of the product is commenced by the introduction of distilled water as the granulation fluid. An atomising pressure of 2 bar is used.

Once the material is granulated, the addition of the granulation fluid is stopped and the powder bulk is dried. The end point of the drying process is indicated by a constant temperature within the powder bed. At this point the temperature of the inlet air is reduced to 25° C. and the bulk material removed. Once cooled the material is screened through a 250 microns sieve and air jet sieved to remove particles below 100 microns. 156

Step 2: Spray Coating With Sureteric

Before applying the Sureteric coat, a 10% w/w dispersion of Opadry II (white) is applied to the granulated Indomethacin to a 2% wt. gain. The 10% w/w dispersion of Opadry II is prepared as follows:

The necessary quantity of Opadry II film coating system is accurately weighed out.

The necessary quantity of water is accurately weighed into the mixing vessel.

With the propeller in the centre and as close to the bottom of the vessel as possible, the water is stirred to form a vortex without drawing air into the liquid.

The Opadry II powder is steadily added to the vortex, avoiding powder flotation on the liquid surface.

The stirrer speed is increased in order to maintain the vortex as required.

After all the Opadry II system is added, the mixer speed is reduced to nearly eliminate the vortex. The dispersion is then mixed for a further 45 minutes.

With the precision coater module attached the vessel is preheated at 70° C. for 15 minutes with a nominal airflow of 6.0 m³/Hr. 60 g of the granulated Indomethacin is returned to the MP Micro and the process temperature set at 75° C. to achieve a product temperature of 40-45° C. The airflow is increased until the product is fluidised. Once the material is equilibrated within the vessel, a constant temperature is reached within the powder bed. The 10% w/w dispersion is then sprayed onto the Indomethacin granules at a rate of 0.2 g/min with an atomising air pressure of 2 bar. Once the desired weight of Opadry II is applied to the granules the pump and the atomising air are stopped and the material is dried until the powder bed reaches a constant temperature. At this point the inlet air temperature is reduced to 25° C. and the operation stopped.

A15% w/w Sureteric dispersion (containing 0.33% w/w simethicone, as an anti-foaming agent) is prepared as follows:

The necessary quantity of Sureteric powder is accurately weighed out.

The necessary quantity of water is accurately weighed into the mixing vessel.

With the propeller in the centre and as close to the bottom of the vessel as possible, the water is stirred to form a vortex without drawing air into the liquid.

The necessary quantity of anti-foaming emulsion is weighed out and added to the water.

The Sureteric powder is steadily added to the vortex, whilst maintaining a vigorous vortex.

The mixer speed is reduced to nearly eliminate the vortex and the dispersion mixed for a further 45 minutes.

Prior to coating, the dispersion is passed through a 250 micron sieve.

Residual Opadry II dispersion is removed from the spray nozzle by rapidly flushing through with the 10% w/w dispersion of Sureteric. The precision coater module is washed and then dried by heating at 85° C. for 15 minutes with a nominal airflow of 6.0 m³/Hr. The Opadry II coated granules are placed into the precision coater and are fluidised using the same conditions as for the addition of Opadry II. Once the material is equilibrated within the vessel, a constant temperature is reached within the powder bed. At this point the granules are coated with the 10% w/w dispersion of Sureteric at a rate of 1.0 g/min. Once a 10-20% wt. gain coating is applied, spraying of the Sureteric dispersion is stopped and the material dried until a constant temperature is observed within the powder bed. At this point the airflow temperature is reduced to 25° C. and the bulk material removed, allowing it to cool.

Step 3: Overcoating With LustreClear

A 9% w/w dispersion of LustreClear is prepared as follows:

The necessary quantity of LustreClear film coating system is accurately weighed out.

The necessary quantity of water is accurately weighed into the mixing vessel.

With the propeller in the centre and as close to the bottom of the vessel as possible, the water is stirred to form a vortex without drawing air into the liquid.

The LustreClear powder is steadily added to the vortex, avoiding powder flotation on the liquid surface.

The stirrer speed is increased in order to maintain the vortex as required.

After all the LustreClear is added, the dispersion is then mixed for a further 3 hours.

The dispersion is then left for a further 2 hours before use.

Residual Sureteric is removed from the spray nozzle by rapidly flushing through with the 9% w/w dispersion of LustreClear. The precision coater module is washed and then dried by heating at 85° C. for 15 minutes with a nominal airflow of 6.0 m³/Hr. The enteric-coated granules are placed into the precision coater and are fluidised using the same conditions as for the addition of Sureteric. Once the material is equilibrated within the vessel, a constant temperature is reached within the powder bed. At this point the granules are coated with the 9% w/w dispersion of LustreClear at a rate of 1.0 g/min. Once a coating of 4-30% wt. gain is applied, spraying of the LustreClear dispersion is stopped and the material dried until a constant temperature is observed within the powder bed. At this point the airflow temperature is reduced to 25° C. and the bulk material removed, allowing it to cool.

### EXAMPLE 3

### Controlled-Release Clarithromycin

Step 1: Granulation of Clarithromycin

Prior to commencing granulation of the Clarithromycin, the vessel of the MP Micro is pre-warmed by heating at 70° C. for 15 minutes with a nominal airflow of 6.0 m³/Hr. 76 g of Clarithromycin and 4 g of PVP K-30 is added to the vessel and the process temperature set to 70° C. The airflow is then increased until the product is fluidised. Once a constant temperature is achieved within the powder bed, spray granulation of the product is commenced by the introduction of distilled water as the granulation fluid. An atomising pressure of 2 bar is used.

Once the material is granulated the addition of the granulation fluid is stopped and the powder bulk is dried. The end point of the drying process is indicated by a constant temperature within the powder bed. At this point the temperature of the inlet air is reduced to 25° C. and the bulk material removed. Once cooled the material is screened through a 250 micron sieve then air jet sieved to remove particles below 100 microns.

Step 2: Spray Coating With Combined Eudragit RS/RL-100

An aqueous dispersion of Eudragit RS/RL-100 is prepared by reconstituting both materials separately as follows:
The necessary quantity of Eudragit is accurately weighed out, necessary to prepare a 12.5% w/w aqueous dispersion.
The necessary quantity of water is accurately weighed into the mixing vessel.
With the propeller in the centre and as close to the bottom of the vessel as possible, the water is stirred to form a vortex without drawing air into the liquid.
The Eudragit powder is steadily added to the vortex, avoiding powder floatation on the liquid surface.
The stirrer speed is increased in order to maintain the vortex as required.
Once all of the Eudragit is added, the mixer speed is reduced to nearly eliminate the vortex. The dispersion is then mixed for a further 120 minutes.
The dispersion is then diluted further by the addition of 10-25% of a suitable plasticiser (in this case Triethyl Citrate)

Once the Eudragit RS-100 and RL-100 is prepared, they are mixed at varying ratios (e.g. 1:3, 1:1 and 3:1) to produce the required release profile. With the precision coater module attached the vessel is preheated at 70° C. for 15 minutes with a nominal airflow of 6.0 m³/Hr. 60 g of the granulated Clarithromycin is returned to the. MP Micro and the process temperature set at 95° C. The airflow is increased until the product is fluidised. Once the material is equilibrated within the vessel, a constant temperature is reached within the powder bed. The material is then sprayed with the Eudragit RS/RL-100 dispersion to achieve a 6-30% wt. gain depending on the desired release profile at a spraying rate of 1.0 g/min with an atomising air pressure of 2 bar.

Once the desired weight of Eudragit coating is added to the granules, the pump and the atomising air are stopped and the material dried until the powder bed reaches a constant temperature. At this point the inlet air temperature is reduced to 25° C. and the operation stopped.

Step 3: Overcoating With LustreClear

A 9% w/w dispersion of LustreClear is prepared as follows:
The necessary quantity of LustreClear film coating system is accurately weighed out.
The necessary quantity of water is accurately weighed into the mixing vessel.
With the propeller in the centre and as close to the bottom of the vessel as possible, the water is stirred to form a vortex without drawing air into the liquid.
The LustreClear powder is steadily added to the vortex, avoiding powder flotation on the liquid surface.
The stirrer speed is increased in order to maintain the vortex as required.
After all the LustreClear is added, the dispersion is then mixed for a further 3 hours.
The dispersion is then left for a further 2 hours before use.

Residual Eudragit is removed from the spray nozzle by rapidly flushing through with the 9% w/w dispersion LustreClear. The precision coater module is washed and then dried by heating at 85° C. for 15 minutes with a nominal airflow of 6.0 m³/Hr. The Eudragit coated granules are placed into the precision coater and are fluidised using the same conditions as for the addition of Eudragit. Once the material is equilibrated within the vessel, a constant temperature is reached within the powder bed. At this point the granules are coated with the 9% w/w dispersion of LustreClear at a rate of 1.0 g/min. Once a coating of 4-30% wt. gain is applied spraying of the LustreClear dispersion is stopped and the material dried until a constant temperature is observed within the powder bed. At this point the airflow temperature is reduced to 25° C. and the bulk material removed, allowing it to cool.

### EXAMPLE 4

### Controlled-Release Enteric-Coated Clarithromycin

Step 1: Granulation of Clarithromycin

Prior to commencing granulation of the Clarithromycin, the vessel of the MP Micro is pre-warmed by heating at 70° C. for 15 minutes with a nominal airflow of 6.0 m³/Hr. 76 g of Clarithromycin and 4 g of PVP K-30 is added to the vessel and the process temperature set to 70° C. The airflow is then increased until the product is fluidised. Once a constant temperature is achieved within the powder bed, spray granulation of the product is commenced by the introduction of distilled water as the granulation fluid. An atomising pressure of 2 bar is used.

Once the material is granulated the addition of the granulation fluid is stopped and the powder bulk is dried. The end point of the drying process is indicated by a constant temperature within the powder bed. At this point the temperature of the inlet air is reduced to 25° C. and the bulk material removed. Once cooled the material is screened through a 250 micron sieve then air jet sieved to remove particles below 100 microns.

Step 2: Spray Coating With Combined Eudragit RS/RL-100

An aqueous dispersion of Eudragit RS/RL-100 is prepared by reconstituting both materials separately as follows:
The necessary quantity of Eudragit is accurately weighed out, necessary to prepare a 12.5% w/w aqueous dispersion.
The necessary quantity of water is accurately weighed into the mixing vessel.
With the propeller in the centre and as close to the bottom of the vessel as possible, the water is stirred to form a vortex without drawing air into the liquid.
The Eudragit powder is steadily added to the vortex, avoiding powder floatation on the liquid surface.
The stirrer speed is increased in order to maintain the vortex as required.
Once all of the Eudragit is added, the mixer speed is reduced to nearly eliminate the vortex. The dispersion is then mixed for a further 120 minutes.
The dispersion is then diluted further by the addition of 10-25% of a suitable plasticiser (in this case Triethyl Citrate)

Once the Eudragit RS-100 and RL-100 is prepared, they are mixed at varying ratios (e.g. 1:3, 1:1 and 3:1) to produce the required release profile. With the precision coater module attached, the vessel is preheated at 70° C. for 15 minutes with a nominal airflow of 6.0 m³/Hr. 60 g of the granulated Clarithromycin is returned to the MP Micro and the process temperature set at 70° C. to achieve a product temperature of 40-45° C. The airflow is increased until the product is fluidised. Once the material is equilibrated within the vessel, a constant temperature is reached within the powder bed. The material is then sprayed with the Eudragit RS/RL-100 dispersion to achieve a 6-30% wt. gain depending on the desired release profile at a spraying rate of 1.0 g/min with an atomising air pressure of 2 bar.

Once the desired weight of Eudragit coating is added to the granules the pump and the atomising air are stopped and the material dried until the powder bed reached a constant temperature. At this point the inlet air temperature is reduced to 25° C. and the operation stopped.

Step 3: Spray Coating With Sureteric

Before applying the Sureteric coat, a 10% w/w dispersion of Opadry II (white) is applied to the granulated Clarithromycin to a 2% wt. gain. The 10% w/w dispersion of Opadry II is prepared as follows:
The necessary quantity of Opadry II film coating system is accurately weighed out.
The necessary quantity of water is accurately weighed into the mixing vessel.
With the propeller in the centre and as close to the bottom of the vessel as possible, the water is stirred to form a vortex without drawing air into the liquid.
The Opadry II powder is steadily added to the vortex, avoiding powder flotation on the liquid surface.
The stirrer speed is increased in order to maintain the vortex as required.
After all the Opadry II system is added, the mixer speed is reduced to nearly eliminate the vortex. The dispersion is then mixed for a further 45 minutes.

With the precision coater module attached the vessel is preheated at 70° C. for 15 minutes with a nominal airflow of 6.0 m³/Hr. 60 g of the granulated Clarithromycin is returned to the MP Micro and the process temperature set at 70° C. to achieve a product temperature of 40-45° C. The airflow is increased until the product is fluidised. Once the material equilibrated within the vessel, a constant temperature is reached within the powder bed. The 10% w/w dispersion is then sprayed onto the Clrithromycin granules at a rate of 1.0 g/min with an atomising air pressure of 2 bar. Once the desired weight of Opadry II is applied to the granules, the pump and the atomising air are stopped and the material dried until the powder bed reached a constant temperature. At this point the inlet air temperature is reduced to 25° C. and the operation stopped.

A 15% w/w Sureteric dispersion (containing 0.33% w/w simethicone, as an anti-foaming agent) is prepared as follows:
The necessary quantity of Sureteric powder is accurately weighed out.
The necessary quantity of water is accurately weighed into the mixing vessel.
With the propeller in the centre and as close to the bottom of the vessel as possible, the water is stirred to form a vortex without drawing air into the liquid.
The necessary quantity of anti-foaming emulsion is weighed out and added to the water.
The Sureteric powder is steadily added to the vortex, whilst maintaining a vigorous vortex.
The mixer speed is reduced to nearly eliminate the vortex and the dispersion is mixed for a further 45 minutes.
Prior to coating, the dispersion is passed through a 250 micron sieve.

Residual Opadry II dispersion is removed from the spray nozzle by rapidly flushing through with the 10% w/w dispersion of Sureteric. The precision coater module is washed and then dried by heating at 85° C. for 15 minutes with a nominal airflow of 6.0 m³/Hr. The Opadry II coated granules are placed into the precision coater and are fluidised using the same conditions as for the addition of Opadry II. Once the material is equilibrated within the vessel, a constant temperature is reached within the powder bed. At this point the granules are coated with the 10% w/w dispersion of Sureteric at a rate of 1.0 g/min. Once a 10-20% wt. gain coating is applied, spraying of the Sureteric dispersion is stopped and the material dried until a constant temperature is observed within the powder bed. At this point the airflow temperature is reduced to 25° C. and the bulk material removed, allowing it to cool.

Step 4: Overcoating With Aquacoat CPD

A 20% w/w dispersion of Aquacoat CPD is prepared as follows:
The necessary quantities of water, Aquacoat CPD and plasticiser (in this case 24% w/w diethyl phthalate) are accurately weighed out.
With the propeller in the centre and as close to the bottom of the vessel as possible, the diethyl phthalate is steadily added to the Aquacoat CPD and mixed for 30 minutes.
The water is then slowly added to the mixture and stirred for a further 10 minutes.

Residual Sureteric dispersion is removed from the spray nozzle by rapidly flushing through with the 20% w/w dispersion of Aquacoat CPD. The precision coater module is washed and then dried by heating at 85° C. for 15 minutes with a nominal airflow of 6.0 m³/Hr. The enteric-coated granules are placed into the precision coater and are fluidised using the same conditions as for the addition of Eudragit. Once the material is equilibrated within the vessel, a constant temperature is reached within the powder bed. At this point the granules are coated with the 20% w/w dispersion of Aquacoat CPD at a rate of 1.5 g/min. Once a coating of 4-30% wt. gain is applied spraying of the Aquacoat CPD dispersion is stopped and the material dried until a constant temperature is observed within the powder bed. At this point the airflow temperature is reduced to 25° C. and the bulk material removed, allowing it to cool.

### EXAMPLE 5

### Taste-Masked Acetaminophen

Step 1: Granulation of Acetaminophen

Prior to commencing granulation of the Acetaminophen, the vessel of the MP Micro is pre-warmed by heating at 70° C. for 15 minutes with a nominal airflow of 6.0 m³/Hr. 76 g of Acetaminophen and 4 g of PVP K-30 is added to the vessel and the process temperature set to 70° C. The airflow is then increased until the product is fluidised. Once a constant temperature is achieved within the powder bed, spray granulation of the product is commenced by the introduction of distilled water as the granulation fluid. An atomising pressure of 2 bar is used.

Once the material is granulated the addition of the granulation fluid is stopped and the powder bulk is dried. The end point of the drying process is indicated by a constant temperature within the powder bed. At this point the temperature of the inlet air is reduced to 25° C. and the bulk material removed. Once cooled the material is screened through a 250 micron sieve and then air jet sieved to remove particles below 100 microns.

Step 2: Spray Coating With Surelease

An aqueous dispersion of Surelease is prepared by diluting to 15% w/w solids (i.e. 60% Surelease dispersion and 40% distilled or deionised water) and stirred using a low shear mixer for approximately 15 minutes. With the precision coater module attached the vessel is preheated at 70° C. for 15 minutes with a nominal airflow of 6.0 m³/Hr. 60 g of the granulated Acetaminophen is returned to the MP Micro and the process temperature set at 70° C. to achieve a product temperature of 40-45° C. The airflow is increased until the product is fluidised. Once the material is equilibrated within the vessel, a constant temperature is reached within the powder bed. The material is then sprayed with the Surelease dispersion to achieve approximately a 15 to 30% wt. gain depending on the degree of tastemasking which is required at a spraying rate of 1.0 g/min with an atomising air pressure of 2 bar.

Once the desired weight of Surelease coating is added to the granules, the pump and the atomising air are stopped and the material is dried until the powder bed reaches a constant temperature. At this point the inlet air temperature is reduced to 25° C. and the operation stopped.

Step 3: Overcoating With a Polyvinylalcohol (PVA) Based Coating System

A 10% w/w dispersion of the PVA based coating system is prepared as follows:
The necessary quantity of the PVA film coating system is accurately weighed out.
The necessary quantity of water is accurately weighed into the mixing vessel.
With the propeller in the centre and as close to the bottom of the vessel as possible, the water is stirred to form a vortex without drawing air into the liquid.
The PVA film coating system is steadily added to the vortex, avoiding powder flotation on the liquid surface.
The stirrer speed is increased in order to maintain the vortex as required.
After all the PVA film coating system is added, the mixer speed is reduced to nearly eliminate the vortex. The dispersion is then mixed for a further 45 minutes.

Residual Surelease is removed from the spray nozzle by rapidly flushing through with the 10% w/w dispersion of the PVA film coating system. The precision coater module is washed and then dried by heating at 85° C. for 15 minutes with a nominal airflow of 6.0 m³/Hr. The Surelease coated granules are placed into the precision coater and are fluidised using the same conditions as for the addition of Surelease. Once the material is equilibrated within the vessel, a constant temperature is reached within the powder bed. At this point the granules are coated with the 10% w/w dispersion of the PVA film coating system at a rate of 1.0 g/min. Once a coating of 4-30% wt. gain is applied spraying of the PVA film coating system is stopped and the material dried until a constant temperature is observed within the powder bed. At this point the airflow temperature is reduced to 25° C. and the bulk material removed, allowing it to cool.

### EXAMPLE 6

### Taste-Masked Verapamil Hydrochloride

Step 1: Granulation of Verapamil

Prior to commencing granulation of the Verapamil, the vessel of the MP Micro is pre-warmed by heating at 70° C. for 15 minutes with a nominal airflow of 6.0 m³/Hr. 76 g of Verapamil and 4 g of PVP K-30 is added to the vessel and the process temperature set to 70° C. The airflow is then increased until the product is fluidised. Once a constant temperature is achieved within the powder bed, spray granulation of the product is commenced by the introduction of distilled water as the granulation fluid. An atomising pressure of 2 bar is used.

Once the material is granulated, the addition of the granulation fluid is stopped and the powder bulk is dried. The end point of the drying process is indicated by a constant temperature within the powder bed. At this point the temperature of the inlet air is reduced to 25° C. and the bulk material removed. Once cooled the material is screened through a 250 micron sieve then air jet sieved to remove particles below 100 microns.

Step 2: Spray Coating With Eudragit RD-100

An aqueous dispersion of Eudragit RD-100 is prepared as follows:
The necessary quantity of Eudragit RD-100 to prepare a 13% w/w dispersion is accurately weighed out.
The necessary quantity of water is accurately weighed into the mixing vessel and 0.003% w/w polysorbate 80 added to it as a plasticiser.
With the propeller in the centre and as close to the bottom of the vessel as possible, the water is stirred to form a vortex without drawing air into the liquid.
The Eudragit RD-100 powder is steadily added to the vortex, avoiding powder flotation on the liquid surface.
The stirrer speed is increased in order to maintain the vortex as required.
Once all of the Eudragit is added, the mixer speed is reduced to nearly eliminate the vortex. The dispersion is then mixed for a further 30 minutes.
The dispersion is then screened through a 0.4 mm mesh prior to use.

With the precision coater module attached the vessel is preheated at 70° C. for 15 minutes with a nominal airflow of 6.0 m³/Hr. 60 g of the granulated Verapamil is returned to the MP Micro and the process temperature set at 95° C. The airflow is increased until the product is fluidised. Once the material is equilibrated within the vessel, a constant temperature is reached within the powder bed. The material is then sprayed with the Eudragit RD-100 dispersion to achieve approximately a 10-15% wt. gain depending on the degree of tastemasking which is required at a spraying rate of 1.0 g/min with an atomising air pressure of 2 bar.

Once the desired weight of Eudragit RD-100 is added to the granules, the pump and the atomising air are stopped and the material dried until the powder bed reaches a constant temperature. At this point the inlet air temperature is reduced to 25° C. and the operation stopped.

Step 3: Overcoating With Neutralised Carbopol 971

A 0.5% w/w aqueous dispersion of neutralised Carbopol 971 is prepared as follows
The necessary quantity of Carbopol 971 to prepare a 0.5% aqueous dispersion is accurately weighed out.
A 0.0025M dispersion of hydrochloric acid is prepared and the necessary quantity weighed into the mixing vessel.
With the propeller in the centre and as close to the bottom of the vessel as possible, the 0.0025M dispersion of hydrochloric acid is stirred to form a vortex without drawing air into the liquid.
The Carbopol 971 powder is steadily added to the vortex, avoiding powder flotation on the liquid surface.
The stirrer speed is increased in order to maintain the vortex as required.
Once all of the Carbopol 971 is added dispersion is mixed for a further 15-20 minutes or until the polymer is swelled to produce a smooth product.

Residual Eudragit RD-100 is removed from the spray nozzle by rapidly flushing through with the dispersion of neutralised Carbopol 971. The precision coater module is washed and then dried by heating at 85° C. for 15 minutes with a nominal airflow of 6.0 m³/Hr. The tastemasked granules are placed into the precision coater and are fluidised using the same conditions as for the addition of Eudragit RD-100. Once the material is equilibrated within the vessel, a constant temperature is reached within the powder bed. At this point the granules are coated with the 0.5% w/w dispersion of neutralised Carbopol 971 at a rate of 10 g/min. Once a coating of 5-30% wt. gain is applied, spraying of the neutralised Carbopol 971 dispersion is stopped and the material dried until a constant temperature is observed within the powder bed. At this point the airflow temperature is reduced to 25° C. and the bulk material removed, allowing it to cool.

### EXAMPLE 7

### Taste-Masked Amoxycillin

Step 1: Granulation of Amoxycillin

Prior to commencing granulation of the Amoxycillin, the vessel of the Mp Micro is pre-warmed by heating at 70° C. for 15 minutes with a nominal airflow of 6.0 m³/Hr. 76 g of Amoxycillin and 4 g of PVP K-30 is added to the vessel and the process temperature set to 50° C. The airflow is then increased until the product is fluidised. Once a constant temperature is achieved within the powder bed, spray granulation of the product is commenced by the introduction of 96% ethanol as the granulation fluid. An atomising pressure of 2 bar is used.

Once the material is granulated the addition of the granulation fluid is stopped and the powder bulk is dried. The end point of the drying process is indicated by a constant temperature within the powder bed. At this point the temperature of the inlet air is reduced to 25° C. and the bulk material removed. Once cooled, the material is screened through a 250 micron sieve and air jet sieved to remove particles below 100 microns.

Step 2: Spray Coating With Opadry AMB

Due to the moisture sensitivity of the Amoxycillin granulation, a moisture barrier film is applied to the material to a 5-30% wt. gain with a 20% w/w dispersion of Opadry AMB, which is prepared as follows:
The necessary quantity of Opadry AMB is accurately weighed out.
The necessary quantity of water is accurately weighed into the mixing vessel.
With the propeller in the centre and as close to the bottom of the vessel as possible, the water is stirred to form a vortex without drawing air into the liquid.
The Opadry AMB powder is steadily added to the vortex, avoiding powder flotation on the liquid surface.
The stirrer speed is increased in order to maintain the vortex as required.
After all the Opadry AMB system is added, the mixer speed is reduced to nearly eliminate the vortex. The dispersion is then mixed for a further 45 minutes.

With the precision coater module attached the vessel is preheated at 70° C. for 15 minutes with a nominal airflow of 6.0 m³/Hr. 60 g of the granulated Amoxycillin is returned to the MP Micro and the process temperature set at 70° C. to achieve a product temperature of 40-45° C. The airflow is increased until the product is fluidised. Once the material equilibrated within the vessel, a constant temperature is reached within the powder bed. The 20% w/w dispersion is then sprayed onto the Amoxycillin granules at a rate of 1.0 g/min with an atomising air pressure of 2.5 bar. Once the desired weight of Opadry AMB is applied to the granules, the pump and the atomising air are stopped and the material dried until the powder bed reaches a constant temperature. At this point the inlet air temperature is reduced to 25° C. and the operation stopped. Once the moisture barrier coating is applied to the granules it is possible to add the functional tastemasking coat to the Amoxycillin.

Step 2: Overcoating With Surelease

An aqueous dispersion of Surelease is prepared by diluting to 15% w/w solids (i.e. 60% Surelease dispersion and 40% distilled or deionised water) and stirred using a low shear mixer for approximately 15 minutes. With the precision coater module attached the vessel is preheated at 70° C. for 15 minutes with a nominal airflow of 6.0 m³/Hr. 60 g of the granulated Amoxycillin is returned to the MP Micro and the process temperature set at 70° C. to achieve a product temperature of 40-45° C. The airflow is increased until the product is fluidised. Once the material is equilibrated within the vessel, a constant temperature is reached within the powder bed. The material is then sprayed with the Surelease dispersion to achieve approximately a 15-30% wt. gain depending on the degree of tastemasking which is required at a spraying rate of 11.0 g/min with an atomising air pressure of 2 bar.

Once the desired weight of Surelease coating is added to the granules the pump and the atomising air are stopped and the material dried until the powder bed reaches a constant temperature. At this point the inlet air temperature is reduced to 25° C. and the operation stopped.

### EXAMPLE 8

### Enteric-Coated Mesalazine

Step 1: Granulation of Mesalazine

Prior to commencing granulation of the Mesalazine, the vessel of the MP Micro is pre-warmed by heating at 70° C. for 15 minutes with a nominal airflow of 6.0 m³/Hr. 76 g of Mesalazine and 4 g of PVP K-30 is added to the vessel and the process temperature set to 70° C. The airflow is then increased until the product is fluidised. Once a constant temperature is achieved within the powder bed, spray granulation of the product is commenced by the introduction of distilled water as the granulation fluid. An atomising pressure of 2 bar is used.

Once the material is granulated, the addition of the granulation fluid is stopped and the powder bulk is dried. The end point of the drying process is indicated by a constant temperature within the powder bed. At this point the temperature of the inlet air is reduced to 25° C. and the bulk material removed. Once cooled the material is screened through a 250 micron sieve and air jet sieved to remove particles below 100 microns.

Step 2: Spray-Coating With Aquacoat CPD

A 20% w/w dispersion of Aquacoat CPD is prepared as follows:
The necessary quantities of water, Aquacoat CPD and plasticiser (in this case 24% w/w diethyl phthalate) are accurately weighed out.
With the propeller in the centre and as close to the bottom of the vessel as possible, the diethyl phthalate is steadily added to the Aquacoat CPD and mixed for 30 minutes.
The water is then slowly added to the mixture and stirred for a further 10 minutes.

With the precision coater module attached the vessel is preheated at 70° C. for 15 minutes with a nominal airflow of 6.0 m³/Hr. 60 g of the granulated Mesalazine is returned to the Mp Micro and the process temperature set at 70° C. to achieve a product temperature of 40-45° C. The airflow is increased until the product is fluidised. Once the material equilibrated within the vessel, a constant temperature is reached within the powder bed. The 20% w/w dispersion is then sprayed onto the Mesalazine granules at a rate of 1.0 g/min with an atomising air pressure of 2.0 bar. Once the desired weight of Aquacoat CPD is applied to the granules, the pump and the atomising air are stopped and the material dried until the powder bed reaches a constant temperature. At this point the inlet air temperature is reduced to 25° C. and the operation stopped.

Step 3: Overcoating With Xanthan Gum

A 5% w/w dispersion of Xanthan Gum is prepared as follows:
The necessary quantity of Xanthan Gum is accurately weighed out.
The necessary quantity of water is accurately weighed into the mixing vessel.
With the propeller in the centre and as close to the bottom of the vessel as possible, the water is stirred to form a vortex without drawing air into the liquid.
The Xanthan Gum powder is steadily added to the vortex, avoiding powder flotation on the liquid surface.
The stirrer speed is increased in order to maintain the vortex as required.
After all the Xanthan Gum system is added, the mixer speed is reduced to nearly eliminate the vortex. The dispersion is then mixed for a further 45 minutes.

Residual Aquacoat CPD is removed from the spray nozzle by rapidly flushing through with the 5% w/w dispersion of Xanthan Gum. The precision coater module is washed and then dried by heating at 85° C. for 15 minutes with a nominal airflow of 6.0 m³/Hr. The enteric coated granules are placed into the precision coater and are fluidised using the same conditions as for the addition of Aquacoat CPD dispersion. Once the material is equilibrated within the vessel, a constant temperature is reached within the powder bed. At this point the granules are coated with the 5% w/w dispersion of Xanthan Gum at a rate of 1.0 g/min. Once a coating of 5-30% wt. gain is applied, spraying of the Xanthan gum dispersion is stopped and the material dried until a constant temperature is observed within the powderbed. At this point the airflow temperature is reduced to 25° C. and the bulk material removed, allowing it to cool.

### EXAMPLE 9

### Controlled-Release Sodium Valproate

Step 1: Granulation of Sodium Valproate

Prior to commencing granulation of the Sodium Valproate, the vessel of the MP Micro is pre-warmed by heating at 70° C. for 15 minutes with a nominal airflow of 6.0 m³/Hr. 76 g of Sodium Valproate and 4 g of PVP K-30 is added to the vessel and the process temperature set to 70° C. The airflow is then increased until the product is fluidised. Once a constant temperature is achieved within the powder bed, spray granulation of the product is commenced by the introduction of distilled water as the granulation fluid. An atomising pressure of 2 bar is used. Once the material is granulated the addition of the granulation fluid is stopped and the powder bulk is dried. The end point of the drying process is indicated by a constant temperature within the powder bed. At this point the temperature of the inlet air is reduced to 25° C. and the bulk material removed. Once cooled the material is screened through a 250 micron sieve and air jet sieved to remove particles below 100 microns.

Step 2: Spray Coating With Surelease

An aqueous dispersion of Surelease is prepared by diluting to 15% w/w solids (i.e. 60% Surelease dispersion and 40% distilled or deionised water) and stirred using a low shear mixer for approximately 15 minutes. With the precision coater module attached the vessel is preheated at 70° C. for 15 minutes with a nominal airflow of 6.0 m³/Hr. 60 g of the granulated Sodium Valproate is returned to the MP Micro and the process temperature set at 70° C. to achieve a product temperature of 40-45° C. The airflow is increased until the product is fluidised. Once the material is equilibrated within the vessel, a constant temperature is reached within the powder bed. The material is then sprayed with the Surelease dispersion to achieve a 6-30% wt. gain depending on the desired release profile at a spraying rate of 1.0 g/min with an atomising air pressure of 2 bar.

Once the desired weight of Surelease coating is added to the granules the pump and the atomising air are stopped and the material dried until the powder bed reached a constant temperature. At this point the inlet air temperature is reduced to 25° C. and the operation stopped.

Stage 3: Overcoating With Eudragit L30 D-55

A plasticized 50% w/w dispersion of Eudragit L30 D-55 formulation for spray coating the Sodium Valproate granules is prepared by diluting to 25% w/w solids with between 5 and 15% w/w plasticizer, 0.2% antifoam agent in distilled or deionised water. The dispersion is then stirred using a low shear mixer for approximately 15 minutes. Prior to use, the plasticized dispersion is filtered through a 0.25 mm sieve. With the precision coater module attached, the vessel is preheated at 70° C. for 15 minutes with a nominal airflow of 6.0 m³/Hr. 60 g of the granulated Sodium Valproate is returned to the MP Micro and the process temperature set at 95° C. The airflow is increased until the product is fluidised. Once the material is equilibrated within the vessel, a constant temperature is reached within the powder bed. The material is then sprayed with the Eudragit dispersion (which is continuously stirred throughout the spraying procedure) to achieve a 8-25% wt. gain depending on the desired degree of mechanical protection which is required at a spraying rate of 1.0 g/min with an atomising air pressure of 2 bar.

Once the desired weight of Eudragit L30 D-55 coating is added to the granules, the pump and the atomising air are stopped and the material dried until the powder bed reached a constant temperature. At this point the inlet air temperature is reduced to 25° C. and the operation stopped.

### EXAMPLE 10

### Wet-Granulated Indomethacin

Step 1: Granulation of Indomethacin

Prior to commencing granulation of the Indomethacin (pulverized), the vessel of an MP Micro fluid bed dryer (available from Niro Pharma Systems of GEA Niro, Inc.) is pre-warmed by heating at 70° C. for 15 minutes with a nominal airflow of 6.0 m³/Hr. 96 g of Indomethacin and 4 g of PVP K-30 is added to the vessel and the process temperature set to 70° C. The airflow is then increased until the product is fluidised. Once a constant temperature is achieved within the powder bed, spray granulation of the product is commenced by the introduction of distilled water as the granulation fluid. An atomising pressure of 2 bar is used.

Once the material was granulated, the addition of the granulation fluid was stopped and the powder bulk was dried. The end point of the drying process is indicated by a constant temperature within the powder bed. At this point the temperature of the inlet air is reduced to 25° C. and the bulk material removed. Once cooled, the material was screened through a 600 micron sieve.

Dissolution testing was then performed using a United States Pharmacopeia Type IV dissolution apparatus (hereinafter USP Type IV apparatus), configured to recirculate the dissolution media. More specifically, the apparatus was a Sotax CE 70. A flow rate of 32 ml/min was used. The drug release was quantified by UV absorbance measured at 318 nm. Dissolution studies were performed in a basic dissolution media (45 minutes pH 6.8 Phosphate Buffer (3:1 ratio of 0.1N HCl:0.2M Na₃PO₄).

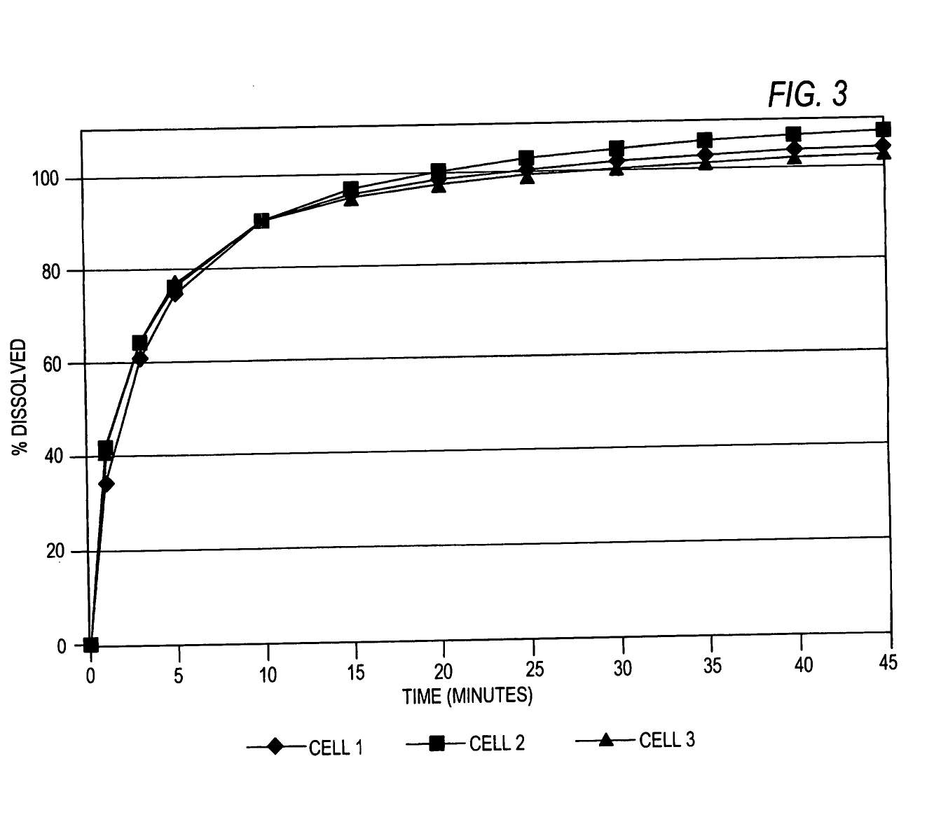

FIG. 3 is a graph plotting the dissolution data for the wet-granulated Indomethacin and 4% PVP K-30 with the pH 6.8 phosphate buffer. The corresponding data plotted in this Figure is shown in the following table:

TABLE 1
% Dissolved

| Time (min) | Cell 1 | Cell 2 | Cell 3 |
|---|---|---|---|
| 0 | 0 | 0 | 0 |
| 1 | 34.46 | 42.04 | 41.12 |
| 3 | 61.21 | 64.43 | 64.7 |
| 5 | 75.16 | 76.27 | 77.04 |
| 10 | 89.9 | 90.23 | 90.04 |
| 15 | 95.55 | 96.55 | 95.01 |
| 20 | 98.62 | 100.27 | 97.6 |
| 25 | 100.45 | 102.71 | 99.28 |
| 30 | 101.84 | 104.47 | 100.46 |
| 35 | 102.86 | 105.95 | 101.44 |
| 40 | 103.65 | 106.94 | 102.22 |
| 45 | 104.36 | 107.73 | 102.97 |

### EXAMPLE 11

Enteric Coated Melt Granulated Indomethacin Formulation

Step 1: Melt Granulation of Indomethacin

Using a Mixer-Granulator P1-6 (available from Dionsa Dierks & Soehne GmbH) equipped with a 1 litre jacketed bowl, 180 g of indomethacin (pulverized) was equilibrated at 70° C. for 10 minutes at a mixer speed of 600 rpm. 20 g of powdered polyethylene glycol (PEG) 6000 was added to the bowl. The massing time, impeller and chopper speeds were varied to achieve to the required granule size distribution (in this case, 100-400 microns in diameter). Once granulated, the material was cooled by reducing the temperature of the bowl jacket to 25° C. whilst mixing at a speed of 100 rpm and a chopper speed of 50 rpm. The mixing continued until the temperature of the powder bed stabilized to around the temperature of the jacketed bowl.

Dissolution testing was then performed using the USP Type IV apparatus described above in connection with Example 10. A flow rate of 32 ml/min was used. The drug release was quantified by UV absorbance measured at 318 nm. Dissolution studies were performed in basic dissolution media (45 minutes pH 6.8 Phosphate Buffer (3:1 ratio of 0.1N HCl:0.2M Na₃PO₄).

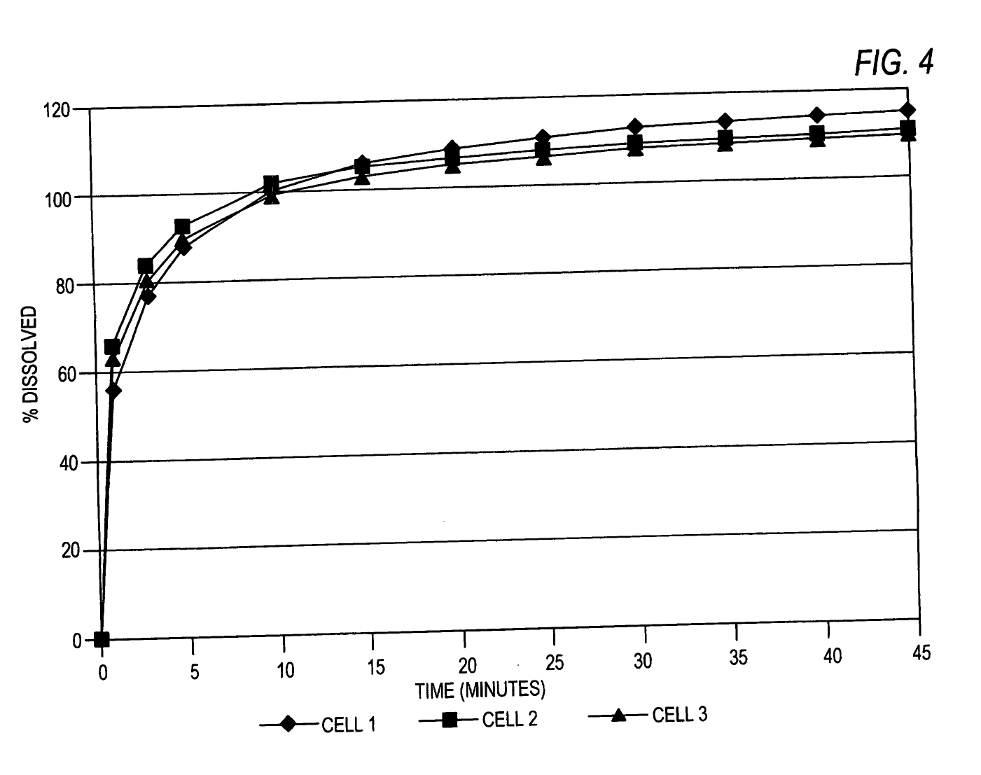

FIG. 4 depicts a graph plotting the dissolution data for the Indomethacin and 10% PEG6000 melt granulation with the pH 6.8 phosphate buffer medium. The corresponding data plotted in this Figure is shown in the following table:

TABLE 2
% Dissolved

| Time (min) | Cell 1 | Cell 2 | Cell 3 |
|---|---|---|---|
| 0 | 0 | 0 | 0 |
| 1 | 55.91 | 65.8 | 63.39 |
| 3 | 77.02 | 83.75 | 80.46 |
| 5 | 87.97 | 92.47 | 89.44 |
| 10 | 100.51 | 101.72 | 99.24 |
| 15 | 106.07 | 105.08 | 103.08 |
| 20 | 108.96 | 106.8 | 104.98 |
| 25 | 110.37 | 107.91 | 106.28 |
| 30 | 112.07 | 108.71 | 107.53 |
| 35 | 113.14 | 109.35 | 108.32 |
| 40 | 113.96 | 109.96 | 108.91 |
| 45 | 114.64 | 110.39 | 109.44 |

It is evident from this data that melt granulating the Indomethacin with PEG 6000 aids the wetting, and hence, the dissolution of the Indomethacin. Specifically, the melt granulated formulation of Example 11 has consistently faster dissolution than the wet granulated formulation of Example 10.
Step 2: Acryl-Eze Enteric Coating of Melt-Granulated Indomethacin and PEG 6000

An aqueous dispersion containing 20% (w/w) Acryl-eze (available from Colorcon) and 0.5% (w/w) simethicone was prepared in an amount sufficient to apply a 15% weight gain of Acryl-eze solids to the indomethacin melt granulation of step 1. The MP-Micro fluid bed drier was used with a Precision Coater Module attached. The Precision Coater Module was preheated at 70° C. for 15 minutes with a nominal airflow of 6.0 m³/Hr. Approximately 100 g of the melt granulated indomethacin of step 1 was loaded into the Precision Coater Module and heated at 60° C. at an airflow sufficient to fluidise the melt granulated indomethacin (hereinafter, the “product”). Once a product temperature of 20°-35° C. was achieved, the product was sprayed with the dispersion of Acryl-eze, until a 15% weight gain was achieved. At this point, the pump and atomising air was stopped, and the sprayed product was dried until the product temperature begins to increase. The inlet air temperature was then reduced to 25° C. and the drying operation was stopped. Any material which had a diameter greater than 600 microns was removed by sieving.

Dissolution testing was then performed using the USP Type IV apparatus described above in connection with Example 10. A flow rate of 32 ml/min was used. The drug release was quantified by UV absorbance measured at 318 nm. Dissolution studies were performed in both acidic dissolution media (0.1N Hydrochloric Acid for 2 hours) and basic dissolution media (45 minutes pH 6.8 Phosphate Buffer (3:1 ratio of 0.1N HCl:0.2M Na₃PO₄).

A concern before preparing an enteric coated, melt granulated formulation was that the acid phase drug release would be unacceptably high, due to a mixing of the enteric coating polymer with the PEG 6000 melt binder. It was postulated that if this occurred, there would be a high degree of drug release in the acid phase due to a dilution of the polymer coat. To prevent this, a melt binder was selected that showed an appreciable difference in melting point (which, for PEG 6000, is 60-65° C.) from the film forming temperature (which, for Acryl-eze, is 25-35° C.) of the enteric coat polymer. It was believed that the mixing of the two materials would thereby be minimised.

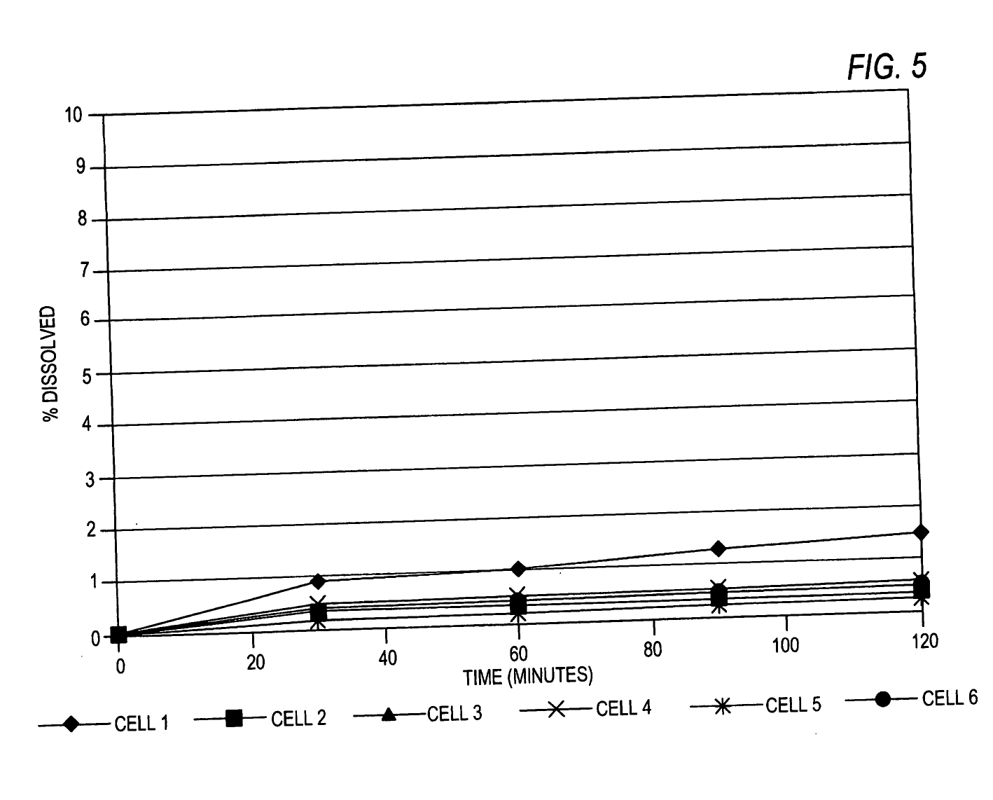

FIG. 5 is a graph plotting the dissolution data for Indomethacin & 10% PEG6000 & 15% Acryl-eze melt granulation prepared in Step 2 with the 0.1N Hydrochloric Acid medium. The corresponding data plotted in this Figure is shown in the following table:

TABLE 3
% Dissolved

| Time (min) | Cell 1 | Cell 2 | Cell 3 | Cell 4 | Cell 5 | Cell 6 |
|---|---|---|---|---|---|---|
| 0 | 0 | 0 | 0 | 0 | 0 | 0 |
| 30 | 0.9 | 0.31 | 0.39 | 0.47 | 0.17 | 0.36 |
| 60 | 1.04 | 0.35 | 0.45 | 0.54 | 0.2 | 0.43 |
| 90 | 1.31 | 0.39 | 0.49 | 0.57 | 0.25 | 0.49 |
| 120 | 1.49 | 0.41 | 0.53 | 0.62 | 0.29 | 0.53 |

It is evident from this data that the enteric coated melt granulated Indomethacin formulation of step 2 does not exhibit a high degree of drug release in the acid phase. To the contrary, less than 1.5% of the formulation dissolved after 2 hours. As such, this formulation meets the U.S.P. acceptance criteria for “Acid Stage” release of “Delayed-release (Enteric-coated) Articles” (less than 10% released in 2 hours in 0.1 N hydrochloric acid in each of 6 units (U.S.P. Level A1)).

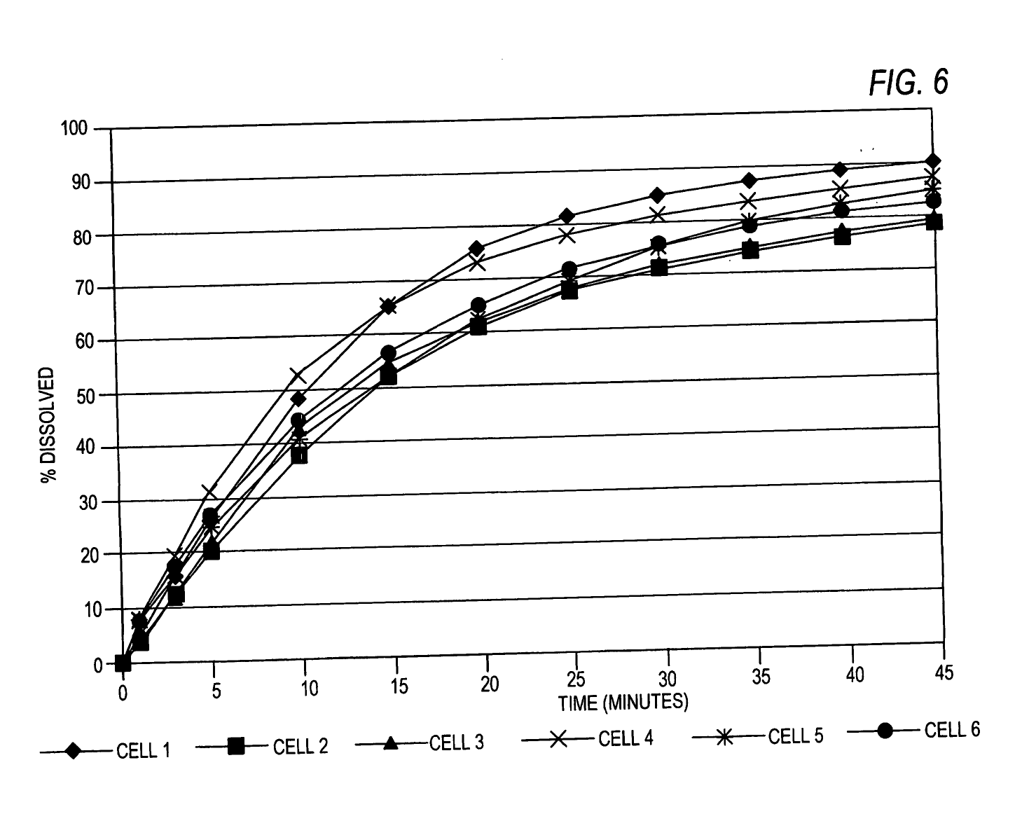

FIG. 6 is a plot of the dissolution data for the Indomethacin & 10% PEG6000 & 15% Acryleze melt granulation from Step 2 with a pH 6.8 phosphate buffer. The corresponding data plotted in this Figure is shown in the following table:

TABLE 4
% Dissolved

| Time (min) | Cell 1 | Cell 2 | Cell 3 | Cell 4 | Cell 5 | Cell 6 |
|---|---|---|---|---|---|---|
| 0 | 0 | 0 | 0 | 0 | 0 | 0 |
| 1 | 4.97 | 3.87 | 3.64 | 8 | 7.85 | 8.16 |
| 3 | 15.54 | 12.34 | 11.68 | 19.13 | 16.04 | 17.43 |
| 5 | 25.49 | 20.08 | 21.91 | 31.1 | 24.63 | 26.73 |
| 10 | 48.14 | 37.55 | 42.88 | 52.55 | 40.93 | 44.06 |
| 15 | 64.99 | 51.69 | 54.43 | 65.13 | 52.12 | 56.24 |
| 20 | 75.34 | 60.83 | 62.12 | 72.9 | 61.78 | 65.1 |
| 25 | 81.23 | 66.99 | 67.36 | 77.55 | 69.09 | 71.12 |
| 30 | 84.62 | 70.83 | 71.53 | 80.95 | 74.95 | 75.38 |
| 35 | 86.89 | 73.62 | 74.52 | 83.31 | 79.1 | 78.37 |
| 40 | 88.54 | 75.98 | 77.06 | 85.09 | 82.18 | 80.62 |
| 45 | 89.67 | 78.19 | 79.2 | 86.58 | 84.48 | 82.3 |

As illustrated in FIG. 6 and Table 4, 78-90% of the indomethacin was released within 45 minutes. As such, this formulation would also appear likely to meet the U.S.P. “Buffer Stage” release of “Delayed-release (Enteric-coated) Articles”. It should be noted that the data does not, in fact pass the Level B1 U.S.P. criteria (80% released within 45 minutes in 6.8 pH buffer in each of 6 units) However, it is believed that the formulation would likely meet the Level B2 U.S.P. criteria (average release at 45 minutes in 6.8 pH buffer for 12 units is at least 75%, with none of the 12 units releasing less than 60% in 45 minutes) and the Level B3 U.S.P. criteria (average release at 45 minutes in 6.8 pH buffer for 24 units is at least 75%, with none of the 24 units releasing less than 50% in 45 minutes, and no more than two of the 24 units releasing less than 60% in 45 minutes). It should be noted that the pH 6.8 buffer phase drug release for this formulation is faster than the corresponding pH 6.8 buffer release in the formulations of Examples 12-15.

### EXAMPLE 12

### Melt-Granulated Sureteric Coated Indomethacin Formulation

Step 1: Sureteric Coating of Indomethacin

An aqueous dispersion containing 15% (w/w) Sureteric (available from Colorcon) and 0.33% (w/w) simethicone was prepared in an amount sufficient to apply a 15% weight gain of Sureteric solids to 100 grams of indomethacin. The MP-Micro fluid bed drier with the Precision Coater Module attached was used. The Precision Coater Module was preheated at 70° C. for 15 minutes with a nominal airflow of 6.0 m³/Hr. The indomethacin (pulverized) was loaded into the Precision Coater Module and heated at 60° C. at an airflow sufficient to fluidise the indomethacin (hereinafter, the “product”). Once a product temperature of 40-45° C. was achieved, the product was sprayed with the dispersion of Sureteric, until a 15% weight gain was achieved. At this point, the pump and atomising air was stopped, and the sprayed product was dried until the product temperature begins to increase. The inlet air temperature was then reduced to 25° C. and the drying operation was stopped. Any material having a diameter greater than 600 microns was removed by sieving.

Step 2 Melt Granulation

Using the Diosna Mixer-Granulator P1-6 equipped with a 1 litre jacketed bowl, 100 g of the material of step 1 was equilibrated at 70° C. for 10 minutes at a mixer speed of 600 rpm. 20 g of powdered polyethylene glycol (PEG) 6000 was added to the bowl. The massing time, impeller and chopper speeds were varied to achieve the required granule size distribution (in this case, 100-400 microns in diameter). Once granulated, the material was cooled by reducing the temperature of the bowl jacket to 25° C. whilst mixing at a speed of 100 rpm and a chopper speed of 50 rpm. The mixing continued until the temperature of the powder bed stabilized to around the temperature of the jacketed bowl.

Dissolution testing was then performed using the USP Type IV apparatus described above in connection with Example 10. A flow rate of 32 ml/min was used. The drug release was quantified by UV absorbance measured at 318 nm. Dissolution studies were performed in both acidic dissolution media (0.1N Hydrochloric Acid for 2 hours) and basic dissolution media (45 minutes pH 6.8 Phosphate Buffer (3:1 ratio of 0.1N HCl: 0.2M Na₃PO₄).

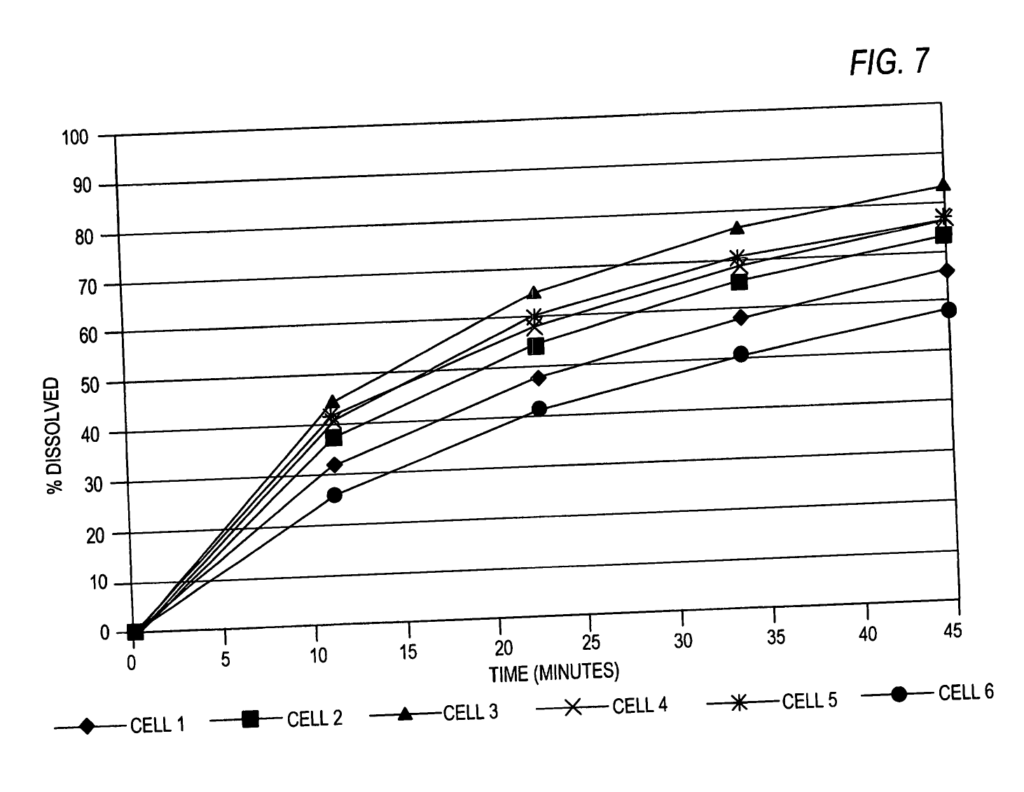

FIG. 7 is a plot of the the dissolution data of the Melt-granulated Sureteric Coated Indomethacin Formulation (Indomethacin and 15% Sureteric and 10% PEG6000) in 0.1 N Hydrochloric acid. The corresponding data plotted in this Figure is shown in the following table:

TABLE 5
% Dissolved

| Time (min) | Cell 1 | Cell 2 | Cell 3 | Cell 4 | Cell 5 | Cell 6 |
|---|---|---|---|---|---|---|
| 0 | 0 | 0 | 0 | 0 | 0 | 0 |
| 30 | 3.18 | 3.74 | 4.47 | 4.14 | 4.04 | 2.58 |
| 60 | 4.75 | 5.41 | 6.49 | 5.8 | 6 | 4.13 |
| 90 | 5.81 | 6.52 | 7.65 | 6.84 | 7.03 | 5.05 |
| 120 | 6.57 | 7.31 | 8.37 | 7.59 | 7.64 | 5.76 |

It is evident from the acid phase release shown in FIG. 7 and Table 5 that Sureteric-coated indomethacin can be melt granulated with PEG6000 without adversely affecting the integrity of the polymer coat. Moreover, the formulation meets the U.S.P. acceptance criteria for “Acid Stage” release of “Delayed-release (Enteric-coated) Articles” (Level A1: less than 10% released in 2 hours in 0.1 N hydrochloric acid in each of 6 units)

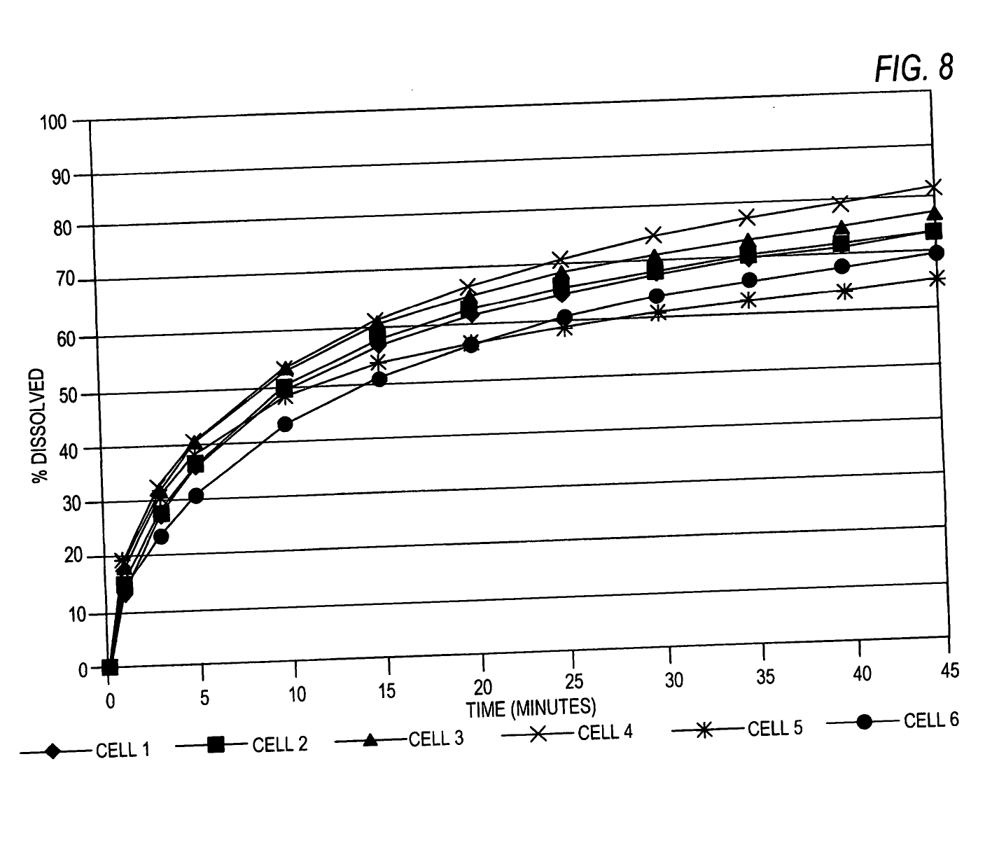

FIG. 8 is a plot of the the dissolution data of the Melt-granulated Sureteric Coated Indomethacin Formulation (Indomethacin and 15% Sureteric and 10% PEG6000) using a pH 6.8 phosphate buffer. The corresponding data plotted in this Figure is shown in the following table:

TABLE 6
% Dissolved

| Time (min) | Cell 1 | Cell 2 | Cell 3 | Cell 4 | Cell 5 | Cell 6 |
|---|---|---|---|---|---|---|
| 0 | 0 | 0 | 0 | 0 | 0 | 0 |
| 1 | 13.15 | 14.8 | 18.27 | 18.68 | 19.35 | 14.02 |
| 3 | 27.14 | 27.59 | 32.03 | 31.92 | 30.51 | 23.47 |
| 5 | 35.82 | 36.15 | 40.53 | 40.26 | 37.8 | 30.49 |
| 10 | 48.9 | 49.8 | 53.32 | 53.07 | 48.2 | 42.78 |
| 15 | 56.46 | 57.52 | 60.46 | 60.91 | 53.41 | 50.6 |
| 20 | 61.4 | 62.35 | 65.16 | 66.64 | 56.42 | 56.12 |
| 25 | 64.94 | 65.72 | 68.46 | 71.05 | 58.62 | 60.22 |
| 30 | 67.58 | 68.18 | 71.11 | 74.51 | 60.48 | 63.25 |
| 35 | 69.62 | 70.25 | 73.2 | 77.17 | 61.97 | 65.67 |
| 40 | 71.33 | 71.78 | 75.08 | 79.38 | 63.24 | 67.64 |
| 45 | 72.81 | 73.12 | 76.71 | 81.28 | 64.45 | 69.24 |

As shown, the total buffer-phase drug release for melt granulated Sureteric-coated indomethacin is slower than the Acryl-eze coated melt granulated indomethacin of Example 11. In particular, only one of the six cells reached 80% drug-release in 45 minutes, with an average 45 minute release of 72.93%. It is believed that the slow release may be attributed either to the increased payload on the granules or a deleterious affect on the polymer coat due to the melt granulation process.

### EXAMPLE 13

### Indomethacin Wet Granulation With a 15% Sureteric Enteric Coat

Step 1: Wet Granulation of Indomethacin

Prior to commencing granulation of the Indomethacin (pulverized), the vessel of the MP Micro was pre-warmed by heating at 70° C. for 15 minutes with a nominal airflow of 6.0 m³/Hr. 96 g of Indomethacin and 4 g of PVP K-30 was added to the vessel and the process temperature set to 70° C. The airflow is then increased until the product is fluidised. Once a constant temperature was achieved within the powder bed, spray granulation of the product was commenced by the introduction of distilled water as the granulation fluid. An atomising pressure of 2 bar was used. Once the material was granulated, the addition of the granulation fluid was stopped and the powder bulk was dried. The end point of the drying process was indicated by a constant temperature within the powder bed. At this point the temperature of the inlet air was reduced to 25° C. and the bulk material removed. Once cooled, the material was screened through a 600 micron sieve.

Step 2: Spray Coating of Wet-Granulated Indomethacin

An aqueous dispersion containing 15% (w/w) Sureteric (available from Colorcon) and 0.33% (w/w) simethicone was prepared in an amount sufficient to apply a 15% weight gain of Sureteric solids to 100 grams of indomethacin. The MP-Micro fluid bed drier with the Precision Coater Module attached was used. The Precision Coater Module was preheated at 70° C. for 15 minutes with a nominal airflow of 6.0 m3/Hr. The indomethacin-PVP granulation was loaded into the Precision Coater Module and heated at 60° C. at an airflow sufficient to fluidise the indomethacin-PVP granulation (hereinafter, the “product”). Once a product temperature of 40-45° C. was achieved, the product was sprayed with the dispersion of Sureteric, until a 15% weight gain was achieved. At this point, the pump and atomising air was stopped, and the sprayed product was dried until the product temperature begins to increase. The inlet air temperature was then reduced to 25° C. and the drying operation was stopped. Any material having a diameter greater than 600 microns was removed by sieving.

Dissolution testing was then performed using the USP Type IV apparatus described above in connection with Example 10. A flow rate of 32 ml/min was used. The drug release was quantified by UV absorbance measured at 318 nm. Dissolution studies were performed in both acidic dissolution media (0.1N Hydrochloric Acid for 2 hours) and basic dissolution media (45 minutes pH 6.8 Phosphate Buffer (3:1 ratio of 0.1N HCl:0.2M Na₃PO₄).

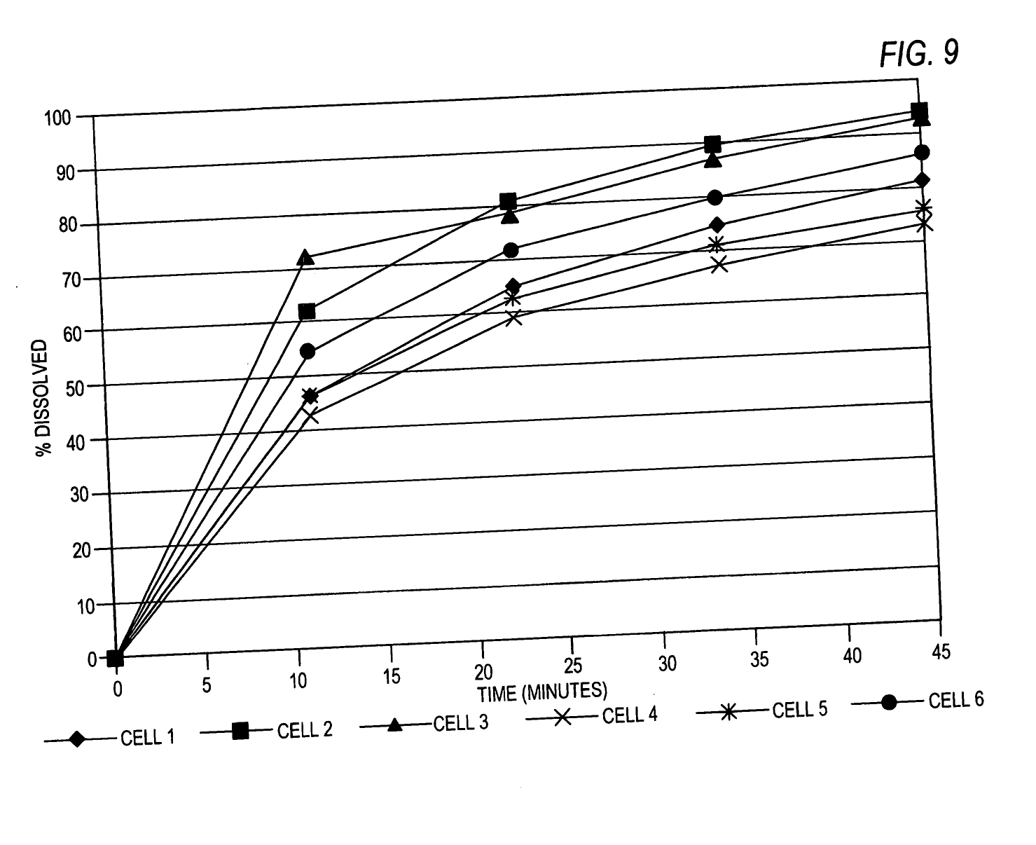

FIG. 9 is a plot of the dissolution data of the Indomethacin Granulation with a 15% Sureteric Enteric Coat (Indomethacin and 15% Sureteric) in 0.1 N Hydrochloric acid. The corresponding data plotted in this Figure is shown in the following table:

TABLE 7
% Dissolved

| Time (min) | Cell 1 | Cell 2 | Cell 3 | Cell 4 | Cell 5 | Cell 6 |
|---|---|---|---|---|---|---|
| 0 | 0 | 0 | 0 | 0 | 0 | 0 |
| 30 | 4.67 | 6.21 | 7.24 | 4.28 | 4.64 | 5.48 |
| 60 | 6.53 | 8.1 | 7.88 | 5.93 | 6.28 | 7.19 |
| 90 | 7.48 | 8.93 | 8.71 | 6.72 | 7.1 | 7.96 |
| 120 | 8.12 | 9.38 | 9.27 | 7.31 | 7.58 | 8.63 |

As such, this formulation meets the U.S.P. acceptance criteria for “Acid Stage” release of “Delayed-release (Enteric-coated) Articles” (less than 10% released in 2 hours in 0.1 N hydrochloric acid in each of 6 units (U.S.P. Level A1)).

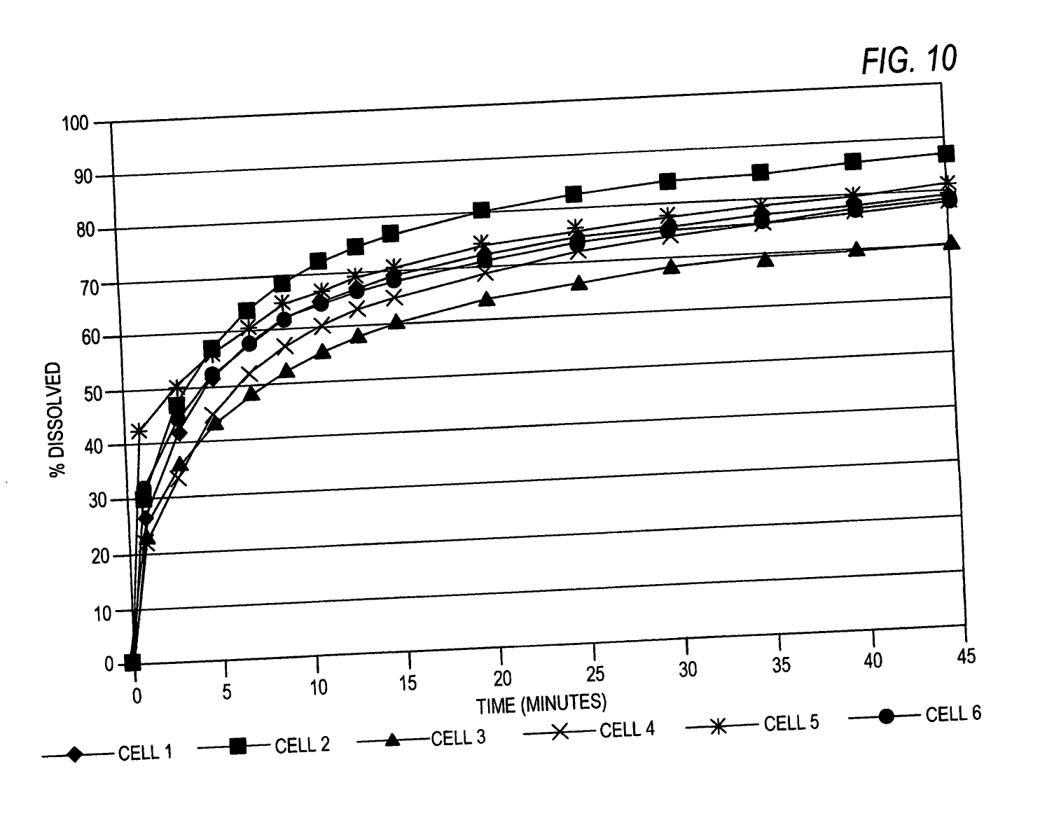

FIG. 10 is a plot of the the dissolution data of Indomethacin Granulation with a 15% Sureteric Enteric Coat in a pH 6.8 phosphate buffer. The corresponding data plotted in this Figure is shown in the following table:

TABLE 8
% Dissolved

| Time (min) | Cell 1 | Cell 2 | Cell 3 | Cell 4 | Cell 5 | Cell 6 |
|---|---|---|---|---|---|---|
| 0 | 0 | 0 | 0 | 0 | 0 | 0 |
| 1 | 26.02 | 29.74 | 23.48 | 21.58 | 42.41 | 31.79 |
| 3 | 42.35 | 46.91 | 36.02 | 33.41 | 50.45 | 44.38 |
| 5 | 51.39 | 56.98 | 43.54 | 44.72 | 56.17 | 52.24 |
| 7 | 57.32 | 63.61 | 48.53 | 51.86 | 60.18 | 57.39 |
| 9 | 61.46 | 68.38 | 52.49 | 56.66 | 64.53 | 61.23 |
| 11 | 64.57 | 71.84 | 55.65 | 60.1 | 66.63 | 64.04 |
| 13 | 66.93 | 74.55 | 58.21 | 62.75 | 68.89 | 66.3 |
| 15 | 68.76 | 76.62 | 60.46 | 64.87 | 70.33 | 68.06 |
| 20 | 72.11 | 80.22 | 64.02 | 68.89 | 73.78 | 71.26 |
| 25 | 74.27 | 82.39 | 66.3 | 71.89 | 75.88 | 73.36 |
| 30 | 75.82 | 83.88 | 68.02 | 74.11 | 77.35 | 74.88 |
| 35 | 77.03 | 85.01 | 69.09 | 75.79 | 78.63 | 76.06 |
| 40 | 78.02 | 85.97 | 69.87 | 77.24 | 79.67 | 77.04 |
| 45 | 78.87 | 86.61 | 70.41 | 78.48 | 80.68 | 77.84 |

As illustrated by the data in FIG. 10 and Table 8, this formulation would also appear likely to meet the U.S.P. “Buffer Stage” release of “Delayed-release (Enteric-coated) Articles”. It should be noted that the data does not, in fact pass the Level B1 U.S.P. criteria (80% released within 45 minutes in 6.8 pH buffer in each of 6 units) However, it is believed that the formulation would likely meet the Level B2 U.S.P. criteria (average release at 45 minutes in 6.8 pH buffer for 12 units is at least 75%, with none of the 12 units releasing less than 60% in 45 minutes) and the Level B3 U.S.P. criteria (average release at 45 minutes in 6.8 pH buffer for 24 units is at least 75%, with none of the 24 units releasing less than 50% in 45 minutes, and no more than two of the 24 units releasing less than 60% in 45 minutes).

### EXAMPLE 14

### Sureteric and LustreClear Coated Indomethacin

Steps 1 and 2: Sureteric Coating of Indomethacin

Indomethacin (pulverized) was coated with Sureteric in the same manner as described in steps 1 and 2 of Example 13.

Step 3: Overcoating With LustreClear

An aqueous dispersion containing 9% (w/w) LustreClear (available from FMC Biopolymer) was prepared in an amount sufficient to apply a 10% weight gain of LustreClear solids to the sureteric coated indomethacin of steps 1 and 2. The MP-Micro fluid bed drier with the Precision Coater Module attached was used. The Precision Coater Module was preheated at 70° C. for 15 minutes with a nominal airflow of 6.0 m³/Hr. The Sureteric Coated Indomethacin was loaded into the Precision Coater Module and heated at 60° C. at an airflow sufficient to fluidise the indomethacin (hereinafter, the “product”). Once a product temperature of 40-45° C. was achieved, the product was sprayed with the dispersion of LustreClear, until a 10% weight gain was achieved. At this point, the pump and atomising air was stopped, and the sprayed product was dried until the product temperature began to increase. The inlet air temperature was then reduced to 25° C. and the drying operation was stopped. Any material having a diameter greater than 600 microns was removed by sieving.

Dissolution testing was then performed using the USP Type IV apparatus described above in connection with Example 10. A flow rate of 32 ml/min was used. The drug release was quantified by UV absorbance measured at 318 nm. Dissolution studies were performed in both acidic dissolution media (0.1N Hydrochloric Acid for 2 hours) and basic dissolution media (45 minutes pH 6.8 Phosphate Buffer (3:1 ratio of 0.1 N HCl:0.2M Na₃PO₄).

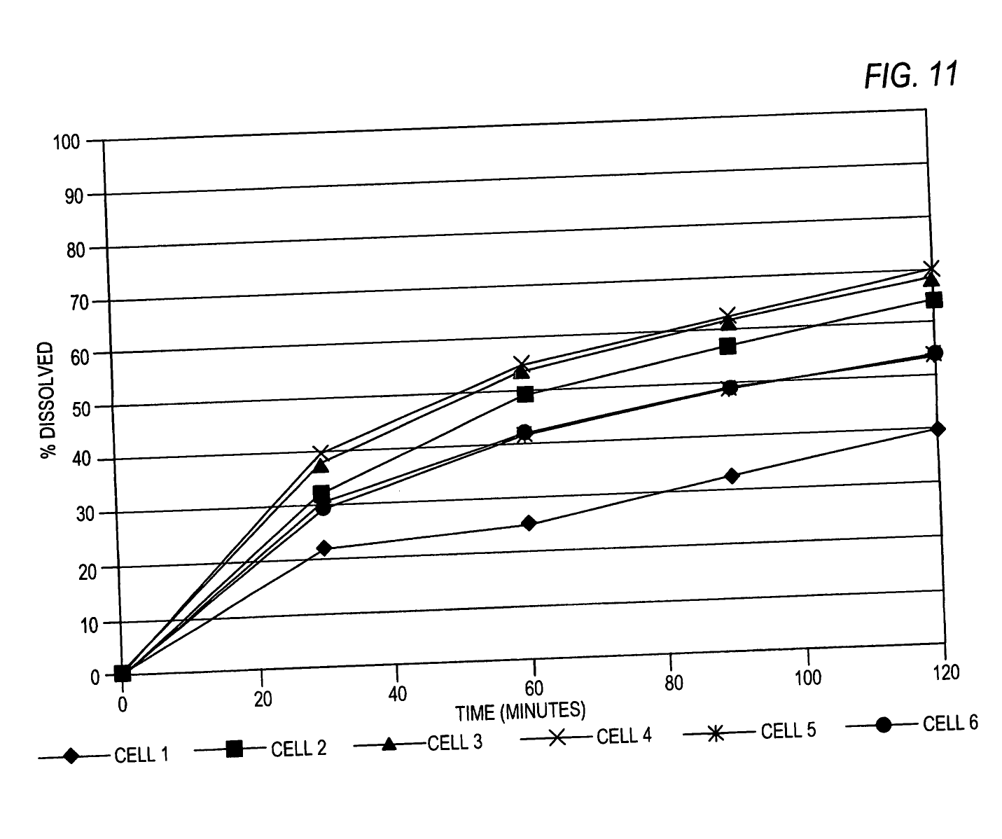

FIG. 11 is a plot of the dissolution data for the Indomethacin and 15% Sureteric with 10% LustreClear in 0.1N Hydrochloric Acid. The corresponding data plotted in this Figure is shown in the following table:

TABLE 9
% Dissolved

| Time (min) | Cell 1 | Cell 2 | Cell 3 | Cell 4 | Cell 5 | Cell 6 |
|---|---|---|---|---|---|---|
| 0 | 0 | 0 | 0 | 0 | 0 | 0 |
| 30 | 2.17 | 3.22 | 3.75 | 3.9 | 3.01 | 2.91 |
| 60 | 2.49 | 4.91 | 5.39 | 5.47 | 4.15 | 4.19 |
| 90 | 3.21 | 5.65 | 6.14 | 6.21 | 4.86 | 4.89 |
| 120 | 3.94 | 6.39 | 6.82 | 6.98 | 5.32 | 5.36 |

It should be noted that the acid-phase drug release of FIG. 11 and Table 9 shows more variability than in the sureteric coated melt-granulation of Example 12, FIG. 7 and Table 5. This suggests that the coating of the PEG6000 of Example 12 may aid the wetting of the particles and hence result in a more reproducible dissolution profile in vitro.

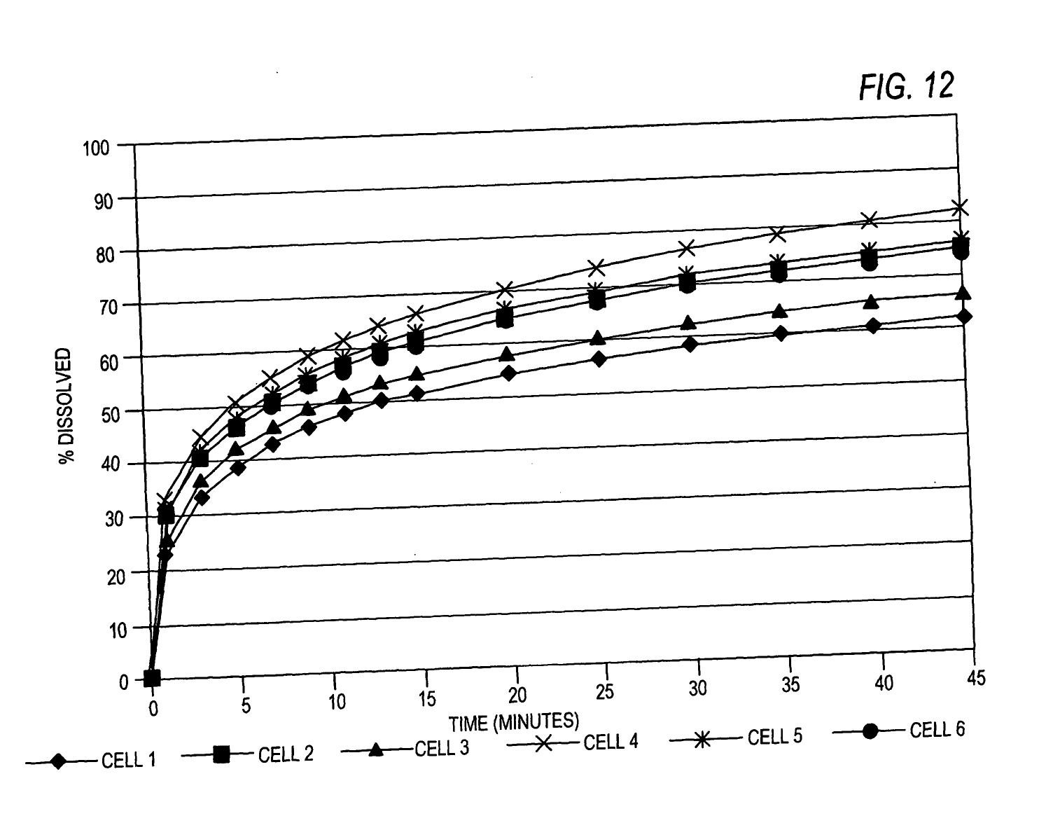

FIG. 12 is a plot of the dissolution data for Indomethacin and 15% Sureteric with 10% LustreClear in the pH 6.8 phosphate buffer. The corresponding data plotted in this Figure is shown in the following table:

TABLE 10
% Dissolved

| Time (min) | Cell 1 | Cell 2 | Cell 3 | Cell 4 | Cell 5 | Cell 6 |
|---|---|---|---|---|---|---|
| 0 | 0 | 0 | 0 | 0 | 0 | 0 |
| 1 | 23.11 | 30.34 | 26.24 | 32.83 | 31.27 | 31 |
| 3 | 33.2 | 40.65 | 37.14 | 44.71 | 42.09 | 40.67 |
| 5 | 38.95 | 46.5 | 42.72 | 50.98 | 47.86 | 46.15 |
| 7 | 42.85 | 50.75 | 46.72 | 55.45 | 52.15 | 50.35 |
| 9 | 45.83 | 54.12 | 49.69 | 58.93 | 55.53 | 53.72 |
| 11 | 48.12 | 56.88 | 52.01 | 61.69 | 58.18 | 55.95 |
| 13 | 50.05 | 59.09 | 54 | 64.05 | 60.52 | 58.15 |
| 15 | 51.61 | 60.94 | 55.58 | 66.14 | 62.5 | 60.07 |
| 20 | 54.69 | 64.7 | 58.72 | 70.38 | 66.43 | 64.41 |
| 25 | 56.88 | 67.7 | 61.01 | 73.67 | 69.38 | 67.24 |
| 30 | 58.48 | 70.02 | 62.8 | 76.39 | 71.62 | 69.61 |
| 35 | 59.81 | 71.93 | 64.28 | 78.63 | 73.38 | 71.19 |
| 40 | 60.93 | 73.54 | 65.55 | 80.64 | 74.86 | 72.71 |
| 45 | 61.88 | 75.01 | 66.7 | 82.37 | 76.17 | 73.88 |

The buffer-phase dissolution profile for this formulation is slow in that only one of the six cells reached 80% drug-release in 45 minutes, with an average 45 minute release of 72.67%. This formulation shows a similar profile to the PEG 6000 melt-granulated, Sureteric-coated indomethacin of Example 12 and FIG. 8.

### EXAMPLE 15

### Lab Scale Melt Granulation of Indomethacin and PEG 6000

The formulation was prepared in the same manner as Example 11, step 1, except that the indomethacin (pulverized) and PEG 600 were mixed in a beaker on a hot-plate using an overhead stirrer, rather than in the Diosna Mixer-Granulator P1-6.

### EXAMPLE 16

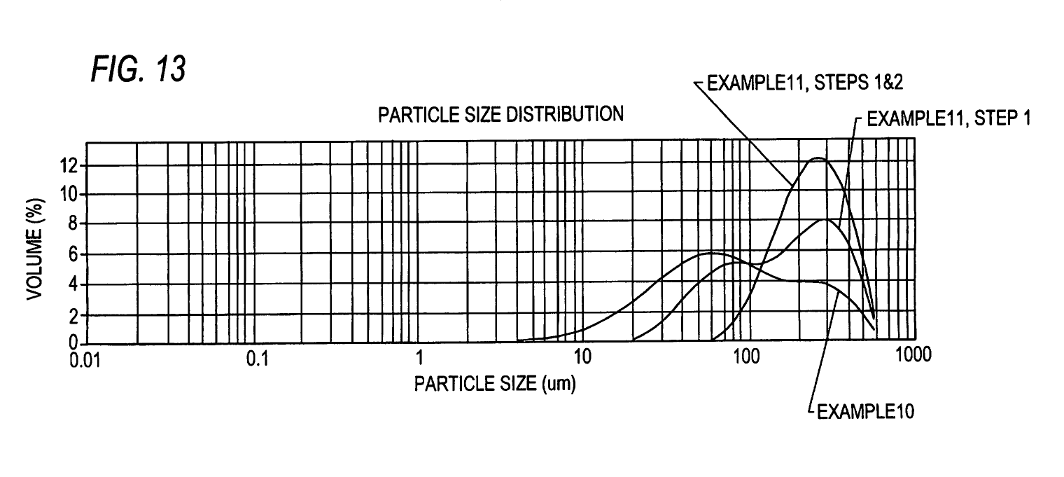

A particle size distribution for the formulations of Examples 10, 11 (steps 1 & 2), and Example 11 (Step 1) is shown in FIG. 13. The data was generated by laser diffraction of particles suspended in an airstream using a Malvern Mastersizer 2000. Referring to FIG. 13, it is shown that the formulation of Example 11 (steps 1 and 2) has the overall largest particle sizes (almost 100% of particles are at least 60 microns), followed the formulation of Example 11 (step 1, only), followed by the formulation of Example 10.

A Twin Stage Impinger Apparatus (glass with a 12.8 mm jet) was used to determine the fine particle fraction of the formulations of Examples 10, 11 (steps 1 & 2), 11 (step 1 only), 14, and 15, with the following results:

| | Average % Fine |
|---|---|
| | Particle Fraction |
| Material | at 601/min (n = 5) |
| 1) Raw indomethacin (pulverized) | 3.89 |
| 2) Indomethacin (pulverized) & 4% PVP K-30 | 0.32 |
| (Example 10) | |
| 3) Indomethacin (pulverized) & 10% PEG 6000 | 0.14 |
| (Example 15) | |
| 4) Indomethacin (pulverized) & 10% PEG 6000 | 0.04 |
| (Example 11, step 1) | |
| 5) Indomethacin (pulverized) & 10% PEG 6000 & | 0.05 |
| 15% Acryl-eze (Example 11, steps 1 and 2) | |
| 6) Indomethacin (pulverized) & 15% Sureteric & | 0.09 |
| 10% LustreClear (Example 14) | |

This fine particle fraction data is consistent with the particle size distribution data of FIG. 13 in that the % Fine Particle Fraction is lowest for Example 11 (steps 1 & 2) and highest for Example 10. Example 14 exhibited a fine particle fraction which was lower than Example 14, but higher than Example 11. The formulation of Example 15 had a fine particle fraction which was lower than Example 10, but higher than Examples 11 and 14. This illustrates that high shear mixing (e.g., with the Dionsna Mixer-Granulator P1-6) produces denser particles having a smaller fine particle fraction than simple mixing in a beaker with an overhead stirrer.

1. A drug formulation for gastrointestinal deposition comprising a non-compressed free flowing plurality of particles comprising a core comprising a drug and a pharmaceutically acceptable excipient, said core overcoated with a functional coating, said drug particles having a mean diameter of greater than 10 μm to about 1 mm, said particles comprising at least about 40% drug.

2. The drug formulation of claim 1 wherein said core comprises drug coated with said excipient and said functional coat overcoats the excipient coat.

3. The drug formulation of claim 1 wherein said core comprises a drug interdispersed in said excipient.

4. The formulation of claim 3 wherein said drug and said excipient are wet granulated.

5. The formulation of claim 3 wherein said drug and said excipient are melt granulated.

6. The formulation of claim 3 wherein said drug and a first portion of said excipient are wet granulated and the resultant wet granulated particles are melt granulated with a second portion of said excipient.

7. The formulation of claim 6 wherein said first portion of excipient and said second portion of excipient comprise the same material.

8. The formulation of claim 6 wherein said first portion of excipient and said second portion of excipient comprise different materials.

9. The formulation of claim 1 wherein said functional coated particles are melt granulated with a pharmaceutically acceptable excipient.

10. The formulation of claim 5 wherein a difference between a film forming temperature of the melt granulating excipient and the film forming temperature of the functional coat is more than 15 degrees C.

11. The formulation of claim 10 wherein the difference between a film forming temperature of a melt granulating excipient and the film forming temperature of a functional coat is more than 20 degrees C.

12. The formulation of claim 11 wherein a difference between a film forming temperature of the melt granulating excipient and a film forming temperature of the functional coat is more than 25 degrees C.

13. The formulation of claim 5 wherein the melt granulating excipient is selected from the group consisting of beeswax, white wax, emulsifying wax, hydrogenated vegetable oil, cetyl alcohol, stearyl alcohol, stearic acid; esters of wax acids; propylene glycol monostearate, glyceryl monostearate; carnauba wax, glyceryl palmitostearate, glyceryl behenate, polyethylene glycol, and a combination thereof.

14. The drug formulation of claim 1 wherein said excipient provides a controlled release of the drug upon gastrointestinal deposition.

15. The drug formulation of claim 14 wherein said excipient provides a controlled release of the drug upon gastrointestinal deposition to provide a therapeutic effect for at least 12 hours after oral administration.

16. The drug formulation of claim 14 wherein said excipient provides a controlled release of the drug upon gastrointestinal deposition to provide a therapeutic effect for at least 24 hours after oral administration.

17. The drug formulation of claim 1 wherein said excipient provides a delayed release of the drug upon gastrointestinal deposition.

18. The drug formulation of claim 17 wherein said excipient provides a delayed release of the drug upon gastrointestinal deposition to effect intestinal absorption.

19. The drug formulation of claim 1 wherein said excipient provides tastemasking.

20. The drug formulation of claim 1 wherein said excipient comprises a salivary stimulant.

21. The drug formulation of claim 2 wherein said excipient provides a moisture barrier.

22. The drug formulation of claim 1 wherein said excipient provides a texture modifier.

23. The drug formulation of claim 1 wherein said functional coating provides a controlled release of the drug upon gastrointestinal deposition.

24. The drug formulation of claim 12 wherein said functional coating provides a controlled release of the drug upon gastrointestinal deposition to provide a therapeutic effect for at least 12 hours after oral administration.

25. The drug formulation of claim 12 wherein said functional coating provides a controlled release of the drug upon gastrointestinal deposition to provide a therapeutic effect for at least 24 hours after oral administration.

26. The drug formulation of claim 1 wherein said functional coating provides a delayed release of the drug upon gastrointestinal deposition.

27. The drug formulation of claim 26 wherein said functional coating provides a delayed release of the drug upon gastrointestinal deposition to effect intestinal absorption.

28. The drug formulation of claim 1 wherein said functional coating provides tastemasking.

29. The drug formulation of claim 1 wherein said functional coating comprises a salivary stimulant.

30. The drug formulation of claim 1 wherein said functional coating provides a moisture barrier.

31. The drug formulation of claim 1 wherein said functional coating provides a texture modifier.

32. The drug formulation of claim 1 wherein said functional coating minimizes asperities on the surface of said particles.

33. The drug formulation of claim 1 wherein said functional coating is resistant to chipping.

34. The drug formulation of claim 1 wherein said functional coating provides pliability to said particles.

35. The drug formulation of claim 1 wherein said drug particles have a mean diameter of greater than about 50 μm.

36. The drug formulation of claim 1 wherein greater than 90% of said particles have a diameter of greater than about 10 μm.

37. The drug formulation of claim 1 wherein greater than 95% of said particles have a diameter of greater than about 10 μm.

38. The drug formulation of claim 1 wherein greater than 99% of said particles have a diameter of greater than about 10 μm.

39. The drug formulation of claim 1 wherein greater than 90% of said particles have a diameter of greater than about 50 μm.

40. The drug formulation of claim 1 wherein greater than 95% of said particles have a diameter of greater than about 50 μm.

41. The drug formulation of claim 1 wherein greater than 99% of said particles have a diameter of greater than about 50 μm.

42. The drug formulation of claim 14 wherein said controlled release excipient is a hydrophobic material.

43. The drug formulation of claim 42 wherein said hydrophobic material is selected from the group consisting of an acrylic polymer, a cellulosic material, shellac, zein and mixtures thereof.

44. The drug formulation of claim 42 wherein said hydrophobic material is an acrylic polymer.

45. The drug formulation of claim 44 wherein said acrylic polymer is selected from the group consisting of acrylic acid and methacrylic acid copolymers, methacrylic acid copolymers, methyl methacrylate copolymers, ethoxyethyl methacrylates, cynaoethyl methacrylate, methyl methacrylate, copolymers, methacrylic acid copolymers, methyl methacrylate copolymers, methyl methacrylate copolymers, methyl methacrylate copolymers, methacrylic acid copolymer, aminoalkyl methacrylate copolymer, methacrylic acid copolymers, methyl methacrylate copolymers, poly(acrylic acid), poly(methacrylic acid, methacrylic acid alkylamide copolymer, poly(methyl methacrylate), poly(methacrylic acid) (anhydride), methyl methacrylate, polymethacrylate, methyl methacrylate copolymer, poly(methyl methacrylate), poly(methyl methacrylate) copolymer, polyacrylamide, aminoalkyl methacrylate copolymer, poly(methacrylic acid anhydride), glycidyl methacrylate copolymers and mixtures thereof.

46. The drug formulation of claim 42 wherein said controlled release excipient is a cellulosic material.

47. The drug formulation of claim 46 wherein said cellulosic material is selected from the group consisting of cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, cellulose acetate butyrate and mixtures thereof.

48. The drug formulation of claim 17 wherein said delayed release material is an enteric polymer.

49. The drug formulation of claim 37 wherein said enteric polymer is selected from the group consisting of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose and mixtures thereof.

50. The drug formulation of claim 19 wherein said tastemasking material is selected from the group consisting of water-soluble sweetening agents, water-soluble artificial sweeteners, dipeptide based sweeteners and mixtures thereof.

51. The drug formulation of claim 50 wherein said water-soluble sweetening agent is selected from the group consisting of monosaccharides, disaccharides and polysaccharides such as xylose, ribose, glucose, mannose, galactose, fructose, dextrose, sucrose, sugar, maltose, partially hydrolyzed starch, or corn syrup solids and sugar alcohols such as sorbitol, xylitol, or mannitol and mixtures thereof.

52. The drug formulation of claim 50 wherein said water-soluble artificial sweetener is selected from the group consisting of soluble saccharin salts, such as sodium or calcium saccharin salts, cyclamate salts, acesulfam-K, the free acid form of saccharin and mixtures thereof.

53. The drug formulation of claim 50 wherein said dipeptide based sweetener is L-aspartyl L-phenylalanine methyl ester.

54. The drug formulation of claim 20 wherein said salivary stimulant is selected from the group consisting of citric acid, tartaric acid, malic acid, fumaric acid, adipic acid, succinic acid, acid anhydrides thereof, acid salts thereof and combinations thereof.

55. The drug formulation of claim 21 wherein said moisture barrier material is selected from the group consisting of acacia gum, acrylic acid polymers and copolymers (polyacrylamides, polyacryldextrans, polyalkyl cyanoacrylates, polymethyl methacrylates), agar-agar, agarose, albumin, alginic acid and alginates, carboxyvinyl polymers, cellulose derivatives such as cellulose acetate, polyamides (nylon 6-10, poly(adipyl-L-lysines, polyterephthalamides and poly-(terephthaloyl-L-lysines)), poly-.epsilon.-caprolactam, polydimethylsiloxane, polyesters, poly(ethylene-vinyl acetate), polyglycolic acid, polyactic acid and its copolymers, polyglutamic acid, polylysine, polystyrene, shellac, xanthan gum, anionic polymers of methacrylic acid and methacrylic acid esters, hydroxyalkylcelluloses and mixtures thereof.

56. The drug formulation of claim 55 wherein said hydroxyalkylcellulose is hydroxypropylmethylcellulose.

57. The drug formulation of claim 22 wherein said texture modifier is selected from the group consisting of acacia gum, acrylic acid polymers and copolymers (polyacrylamides, polyacryldextrans, polyalkyl cyanoacrylates, polymethyl methacrylates), agar-agar, agarose, albumin, alginic acid and alginates, carboxyvinyl polymers, cellulose derivatives such as cellulose acetate, polyamides (nylon 6-10, poly(adipyl-L-lysines, polyterephthalamides and poly-(terephthaloyl-L-lysines)), poly-epsilon.-caprolactam, polydimethylsiloxane, polyesters, poly (ethylene-vinyl acetate), polyglycolic acid, polyactic acid and its copolymers, polyglutamic acid, polylysine, polystyrene, shellac, xanthan gum, anionic polymers of methacrylic acid and methacrylic acid esters, hydroxyalkylcelluloses and mixtures thereof.

58. The drug formulation of claim 32 wherein said particulates have a mean rugosity of from about 1.0 to about 1.5.

59. The drug formulation of claim 33 wherein said chip resistant coating comprises a material selected from the group consisting of acacia gum, alginic acid and alginates, carboxymethylcellulose, ethylcellulose, gelatine, hydroxypropylcellulose, hydroxypropylmethylcellulose, methylcellulose, xanthan gum, pectin, tragacanth, microcrystalline cellulose, hydroxyethylcellulose, ethylhydroxyethylcellulose, sodium carboxymethylcellulose, polyethylene glycols, polyvinylpyrrolidone, polyvinyl alcohol, polyacrylic acid, gum arabic, lactose, starch (wheat, maize, potato and rice starch), sucrose, glucose, mannitol, sorbitol, xylitol, stearic acid, hydrogenated cottonseed oil, hydrogenated castor oil, vinylpyrrolidone-vinyl acetate copolymers, fructose, methylhydroxyethylcellulose, agar-agar, carrageenan, karaya gum, chitosan, starch hydrolysates and mixtures thereof.

60. The drug formulation of claim 34 wherein said pliable coating comprises a plasticizer selected from the group consisting of dibutyl sebacate, diethyl phthalate, triethyl citrate, tibutyl citrate, triacetin and mixtures thereof.

61. A drug delivery system comprising a dosing device comprising a housing and an actuator, said device containing at least one unit dose of a drug formulation according to claim 1, said device upon actuation delivering a unit dose of said drug formulation such that an effective dose of said drug cannot be delivered into the lower lung of a human patient.

62. A drug delivery system comprising a multiple unit dosing device comprising a housing and an actuator, said device containing multiple unit doses of a drug formulation according to claim 1, said device upon actuation delivering a unit dose of said drug formulation such that an effective dose of said drug cannot be delivered into the lower lung of a human patient.

63. A drug delivery system comprising a multiple unit dosing device comprising a housing and an actuator, said device containing at least one unit dose of a drug formulation comprising a non-compressed free flowing plurality of particles comprising a core comprising a drug and a pharmaceutically acceptable excipient, said core overcoated with a functional coating, said drug particles having a mean diameter of greater than 10 μm to about 1 mm, said device upon actuation delivering a unit dose of said drug formulation such that an effective dose of said drug cannot be delivered into the lower lung of a human patient.

64. The formulation of claim 63 wherein said drug and said excipient are wet granulated.

65. The formulation of claim 63 wherein said drug and said excipient are melt granulated.

66. The formulation of claim 63 wherein said drug and a first portion of said excipient are wet granulated and the resultant wet granulated particles are melt granulated with a second portion of said excipient.

67. The formulation of claim 66 wherein said first portion of excipient and said second portion of excipient comprise the same material.

68. The formulation of claim 66 wherein said first portion of excipient and said second portion of excipient comprise different materials.

69. The formulation of claim 63 wherein said functional coated particles are melt granulated with a pharmaceutically acceptable excipient.

70. The formulation of claim 65 wherein a difference between a film forming temperature of the melt granulating excipient and a film forming temperature of a functional coat is more than 15 degrees C.

71. The formulation of claim 70 wherein a difference between a film forming temperature of a melt granulating excipient and a film forming temperature of the functional coat is more than 20 degrees C.

72. The formulation of claim 71 wherein a difference between a film forming temperature point of the melt granulating excipient and a film forming temperature of the functional coat is more than 25 degrees C.

73. The formulation of claim 65 wherein the melt granulating excipient is selected from the group consisting of beeswax, white wax, emulsifying wax, hydrogenated vegetable oil, cetyl alcohol, stearyl alcohol, stearic acid; esters of wax acids; propylene glycol monostearate, glyceryl monostearate; carnauba wax, glyceryl palmitostearate, glyceryl behenate, polyethylene glycol, and a combination thereof.

74. A method of administering a drug to a human patient for gastrointestinal deposition comprising formulating a drug formulation comprising a non-compressed free flowing plurality of particles comprising a core comprising a drug and a pharmaceutically acceptable excipient, said core overcoated with a functional coating, said drug particles having a mean diameter of greater than 10 μm to about 1 mm, containing said drug formulation in a drug delivery device capable of administering multiple unit doses of said multiparticulates into the oral cavity; administering a unit dose of the multiparticulates to the oral cavity wherein greater than about 80% of the unit dose is deposited in the gastrointestinal tract.

75. A method of preparing a drug delivery system for delivering multiple doses of a drug for gastrointestinal deposition comprising preparing a drug formulation comprising a non-compressed free flowing plurality of particles comprising a core comprising a drug and a pharmaceutically acceptable excipient, said core overcoated with a functional coating, said drug particles having a mean diameter of greater than 10 μm to about 1 mm; and placing multiple unit doses of said drug formulation in a device which meters a single unit dose for delivery.

76. The method of claim 74 wherein said core comprises drug coated with said excipient and said functional coat overcoats the excipient coat.

77. The method of claim 74 wherein said core comprises a drug interdispersed in said excipient.

78. The formulation of claim 77 wherein said drug and said excipient are wet granulated.

79. The formulation of claim 77 wherein said drug and said excipient are melt granulated.

80. The formulation of claim 77 wherein said drug and a first portion of said excipient are wet granulated and the resultant wet granulated particles are melt granulated with a second portion of said excipient.

81. The formulation of claim 80 wherein said first portion of excipient and said second portion of excipient comprise the same material.

82. The formulation of claim 80 wherein said first portion of excipient and said second portion of excipient comprise different materials.

83. The formulation of claim 74 wherein said functional coated particles are melt granulated with a pharmaceutically acceptable excipient.

84. The formulation of claim 79 wherein a difference between a film forming temperature of the melt granulating excipient and a film forming temperature of the functional coat is more than 15 degrees C.

85. The formulation of claim 84 wherein a difference between a film forming temperature of the melt granulating excipient and a film forming temperature of the functional coat is more than 20 degrees C.

86. The formulation of claim 85 wherein a difference between a film forming temperature of the melt granulating excipient and a film forming temperature of the functional coat is more than 25 degrees C.

87. The formulation of claim 79 wherein the melt granulating excipient is selected from the group consisting of beeswax, white wax, emulsifying wax, hydrogenated vegetable oil, cetyl alcohol, stearyl alcohol, stearic acid; esters of wax acids; propylene glycol monostearate, glyceryl monostearate; carnauba wax, glyceryl palmitostearate, glyceryl behenate, polyethylene glycol, and a combination thereof.

88. The method of claim 74 wherein said excipient provides a controlled release of the drug upon gastrointestinal deposition.

89. The method of claim 88 wherein said excipient provides a controlled release of the drug upon gastrointestinal deposition to provide a therapeutic effect for at least 12 hours after oral administration.

90. The method of claim 88 wherein said excipient provides a controlled release of the drug upon gastrointestinal deposition to provide a therapeutic effect for at least 24 hours after oral administration.

91. The method of claim 74 wherein said excipient provides a delayed release of the drug upon gastrointestinal deposition.

92. The method of claim 91 wherein said excipient provides a delayed release of the drug upon gastrointestinal deposition to effect intestinal absorption.

93. The method of claim 74 wherein said excipient provides tastemasking.

94. The method of claim 74 wherein said excipient comprises a salivary stimulant.

95. The method of claim 76 wherein said excipient provides a moisture barrier.

96. The method of claim 74 wherein said excipient provides a texture modifier.

97. The method of claim 74 wherein said functional coating provides a controlled release of the drug upon gastrointestinal deposition.

98. The method of claim 97 wherein said functional coating provides a controlled release of the drug upon gastrointestinal deposition to provide a therapeutic effect for at least 12 hours after oral administration.

99. The method of claim 97 wherein said functional coating provides a controlled release of the drug upon gastrointestinal deposition to provide a therapeutic effect for at least 24 hours after oral administration.

100. The method of claim 74 wherein said functional coating provides a delayed release of the drug upon gastrointestinal deposition.

101. The method of claim 100 wherein said functional coating provides a delayed release of the drug upon gastrointestinal deposition to effect intestinal absorption.

102. The method of claim 74 wherein said functional coating provides tastemasking.

103. The method of claim 74 wherein said functional coating comprises a salivary stimulant.

104. The method of claim 74 wherein said functional coating provides a moisture barrier.

105. The method of claim 74 wherein said functional coating provides a texture modifier.

106. The method of claim 74 wherein said functional coating minimizes asperities on the surface of said particles.

107. The method of claim 74 wherein said functional coating is resistant to chipping.

108. The method of claim 74 wherein said functional coating provides pliability to said particles.

109. The system of claim 74 wherein said drug particles have a mean diameter of greater than about 50 μm.

110. The method of claim 74 wherein greater than 90% of said particles have a diameter of greater than about 10 μm.

111. The method of claim 74 wherein greater than 95% of said particles have a diameter of greater than about 10 μm.

112. The method of claim 74 wherein greater than 99% of said particles have a diameter of greater than about 10 μm.

113. The method of claim 74 wherein greater than 90% of said particles have a diameter of greater than about 50 μm.

114. The method of claim 74 wherein greater than 95% of said particles have a diameter of greater than about 50 μm.

115. The method of claim 74 wherein greater than 99% of said particles have a diameter of greater than about 50 μm.

116. The method of claim 88 wherein said controlled release excipient is a hydrophobic material.

117. The method of claim 116 wherein said hydrophobic material is selected from the group consisting of an acrylic polymer, a cellulosic material, shellac, zein and mixtures thereof.

118. The method of claim 116 wherein said hydrophobic material is an acrylic polymer.

119. The method of claim 118 wherein said acrylic polymer is selected from the group consisting of acrylic acid and methacrylic acid copolymers, methacrylic acid copolymers, methyl methacrylate copolymers, ethoxyethyl methacrylates, cynaoethyl methacrylate, methyl methacrylate, copolymers, methacrylic acid copolymers, methyl methacrylate copolymers, methyl methacrylate copolymers, methyl methacrylate copolymers, methacrylic acid copolymer, aminoalkyl methacrylate copolymer, methacrylic acid copolymers, methyl methacrylate copolymers, poly(acrylic acid), poly(methacrylic acid, methacrylic acid alkylamide copolymer, poly(methyl methacrylate), poly(methacrylic acid) (anhydride), methyl methacrylate, polymethacrylate, methyl methacrylate copolymer, poly(methyl methacrylate), poly(methyl methacrylate) copolymer, polyacrylamide, aminoalkyl methacrylate copolymer, poly(methacrylic acid anhydride), glycidyl methacrylate copolymers and mixtures thereof.

120. The method of claim 116 wherein said controlled release excipient is a cellulosic material.

121. The method of claim 120 wherein said cellulosic material is selected from the group consisting of cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, cellulose acetate butyrate and mixtures thereof.

122. The method of claims 91 and 100 wherein said delayed release material is an enteric polymer.

123. The method of claim 122 wherein said enteric polymer is selected from the group consisting of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose and mixtures thereof.

124. The method of claim 93 wherein said tastemasking material is selected from the group consisting of water-soluble sweetening agents, water-soluble artificial sweeteners, dipeptide based sweeteners and mixtures thereof.

125. The drug formulation of claim 124 wherein said water-soluble sweetening agent is selected from the group consisting of monosaccharides, disaccharides and polysaccharides such as xylose, ribose, glucose, mannose, galactose, fructose, dextrose, sucrose, sugar, maltose, partially hydrolyzed starch, or corn syrup solids and sugar alcohols such as sorbitol, xylitol, or mannitol and mixtures thereof.

126. The method of claim 124 wherein said water-soluble artificial sweetener is selected from the group consisting of soluble saccharin salts, such as sodium or calcium saccharin salts, cyclamate salts, acesulfam-K, the free acid form of saccharin and mixtures thereof.

127. The method of claim 124 wherein said dipeptide based sweetener is L-aspartyl L-phenylalanine methyl ester.

128. The method of claim 94 wherein said salivary stimulant is selected from the group consisting of citric acid, tartaric acid, malic acid, fumaric acid, adipic acid, succinic acid, acid anhydrides thereof, acid salts thereof and combinations thereof.

129. The method of claim 95 wherein said moisture barrier material is selected from the group consisting of acacia gum, acrylic acid polymers and copolymers (polyacrylamides, polyacryldextrans, polyalkyl cyanoacrylates, polymethyl methacrylates), agar-agar, agarose, albumin, alginic acid and alginates, carboxyvinyl polymers, cellulose derivatives such as cellulose acetate, polyamides (nylon 6-10, poly(adipyl-L-lysines, polyterephthalamides and poly-(terephthaloyl-L-lysines)), poly-.epsilon.-caprolactam, polydimethylsiloxane, polyesters, poly (ethylene-vinyl acetate), polyglycolic acid, polyactic acid and its copolymers, polyglutamic acid, polylysine, polystyrene, shellac, xanthan gum, anionic polymers of methacrylic acid and methacrylic acid esters, hydroxyalkylcelluloses and mixtures thereof.

130. The method of claim 129 wherein said hydroxyalkylcellulose is hydroxypropylmethylcellulose.

131. The method of claim 96 wherein said texture modifier is selected from the group consisting of acacia gum, acrylic acid polymers and copolymers (polyacrylamides, polyacryldextrans, polyalkyl cyanoacrylates, polymethyl methacrylates), agar-agar, agarose, albumin, alginic acid and alginates, carboxyvinyl polymers, cellulose derivatives such as cellulose acetate, polyamides (nylon 6-10, poly(adipyl-L-lysines, polyterephthalamides and poly-(terephthaloyl-L-lysines)), poly-.epsilon.-caprolactam, polydimethylsiloxane, polyesters, poly (ethylene-vinyl acetate), polyglycolic acid, polyactic acid and its copolymers, polyglutamic acid, polylysine, polystyrene, shellac, xanthan gum, anionic polymers of methacrylic acid and methacrylic acid esters, hydroxyalkylcelluloses and mixtures thereof.

132. The method of claim 116 wherein said particulates have a mean rugosity of from about 1.0 to about 1.5.

133. The drug formulation of claim 77 wherein said chip resistant coating comprises a material selected from the group consisting of acacia gum, alginic acid and alginates, carboxymethylcellulose, ethylcellulose, gelatine, hydroxypropylcellulose, hydroxypropylmethylcellulose, methylcellulose, xanthan gum, pectin, tragacanth, microcrystalline cellulose, hydroxyethylcellulose, ethylhydroxyethylcellulose, sodium carboxymethylcellulose, polyethylene glycols, polyvinylpyrrolidone, polyvinyl alcohol, polyacrylic acid, gum arabic, lactose, starch (wheat, maize, potato and rice starch), sucrose, glucose, mannitol, sorbitol, xylitol, stearic acid, hydrogenated cottonseed oil, hydrogenated castor oil, vinylpyrrolidone-vinyl acetate copolymers, fructose, methylhydroxyethylcellulose, agar-agar, carrageenan, karaya gum, chitosan, starch hydrolysates and mixtures thereof.

134. The method of claim 108 wherein said pliable coating comprises a plasticizer selected from the group consisting of dibutyl sebacate, diethyl phthalate, triethyl citrate, tibutyl citrate, triacetin and mixtures thereof.

135. A method of preparing a multiparticulate drug formulation for gastrointestinal deposition with minimal potential for surface water coalesence comprising preparing a non-compressed free flowing plurality of particles comprising a core comprising a drug and a pharmaceutically acceptable excipient, and overcoating said core with a coating minimizes water coalesence on the surface of said particles.

136. A method of preparing a multiparticulate drug formulation for gastrointestinal deposition with minimal static charge comprising preparing a non-compressed free flowing plurality of particles comprising a core comprising a drug and a pharmaceutically acceptable excipient, and overcoating said core with a coating which minimizes static charge between said particles.

137. A method of preparing a multiparticulate drug formulation for gastrointestinal deposition comprising preparing a non-compressed free flowing plurality of particles comprising a core comprising a drug and a pharmaceutically acceptable excipient air jet sieving said particles to separate said cores from fine particles; and overcoating said core with a functional coating.

138. A method of preparing a multiparticulate drug formulation with improved weight uniformity for gastrointestinal deposition comprising preparing a non-compressed free flowing plurality of particles comprising a core comprising a drug and a pharmaceutically acceptable excipient; and overcoating said core with a functional coating.

139. A method of preparing a multiparticulate drug formulation for gastrointestinal deposition with minimal change in cohesiveness in response to humidity change comprising preparing a non-compressed free flowing plurality of particles comprising a core comprising a drug and a pharmaceutically acceptable excipient; and overcoating said core with a functional coating such that the cohesiveness of said particles does not substantially change over a humidity gradient from about 10% relative humidity to about 90% relative humidity.

140. The method of claim 137 wherein said fine particles are less than about 50 micrometers.

141. The method of claim 137 wherein said fine particles are less than about 25 micrometers.

142. The method of claim 137 wherein said fine particles are less than about 10 micrometers.

143. The method of claim 137 further comprising filtering said particles prior to air jet sieving to remove particles greater than about 500 micrometers.

144. The method of claim 143 further comprising filtering said particles prior to air jet sieving to remove particles greater than about 750 micrometers.

145. The method of claim 144 further comprising filtering said particles prior to air jet sieving to remove particles greater than about 1 mm.

146. The method of claim 135 comprising preparing said particles with an amount of coloring agents which minimizes weakening of the adhesion of the overcoat to the core.

147. The method of claim 146 wherein said coloring agent is selected from the group consisting of a lake, an opacifier or a combination thereof.

148. The method of claim 146 wherein said coloring agent does not comprise a lake.

149. The method of claim 146 wherein said coloring agent does not comprise an opacifier.

150. The method of claim 146 wherein said coloring agent does not comprise a lake or an opacifier.

151. The method of claim 135 wherein said overcoat comprises a plasticizer.

152. The method of claim 139 wherein the cohesiveness of said particles does not substantially change over a humidity gradient from about 20% relative humidity to about 80% relative humidity.

153. The method of claim 152 wherein the cohesiveness of said particles does not substantially change over a humidity gradient from about 40% relative humidity to about 60% relative humidity.

154. The method of claim 137 wherein said overcoat comprises a conductive polymer.

155. The method of claim 135 wherein said drug particles having a mean diameter of greater than 10 μm to about 1 mm.

156. The method of claim 155 wherein said drug particles having a mean diameter of greater than 50 μm to about 500 μm.

157. The method of claim 135 wherein said particles comprise at least about 40%, at least about 50%, at least about 60%, at least about 70% or at least about 80% drug.

158. The method of claim 135 wherein said core comprises drug coated with said excipient and said functional coat overcoats the excipient coat.

159. The method of claim 135 wherein said core comprises a drug interdispersed in said excipient.

160. The method of claim 159 wherein said drug and said excipient are wet granulated.

161. The method of claim 159 wherein said drug and said excipient are melt granulated.

162. The method of claim 159 wherein said drug and a first portion of said excipient are wet granulated and the resultant wet granulated particles are melt granulated with a second portion of said excipient.

163. The method of claim 162 wherein said first portion of excipient and said second portion of excipient comprise the same material.

164. The method of claim 162 wherein said first portion of excipient and said second portion of excipient comprise different materials.

165. The method of claim 135 wherein said functional coated particles are melt granulated with a pharmaceutically acceptable excipient.

166. The method of claim 161 wherein a difference between a film forming temperature of the melt granulating excipient and a film forming temperature of the functional coat is more than 15 degrees C.

167. The method of claim 166 wherein a difference between a film forming temperature of the melt granulating excipient and a film forming temperature of the functional coat is more than 20 degrees C.

168. The method of claim 166 wherein a difference between a film forming temperature of a melt granulating excipient and a film forming temperature of the functional coat is more than 25 degrees C.

169. The method of claim 161 wherein the melt granulating excipient is selected from the group consisting of beeswax, white wax, emulsifying wax, hydrogenated vegetable oil, cetyl alcohol, stearyl alcohol, stearic acid; esters of wax acids; propylene glycol monostearate, glyceryl monostearate; carnauba wax, glyceryl palmitostearate, glyceryl behenate, polyethylene glycol, and a combination thereof.

170. A multiparticulate formulation obtained according to a process of claim 135.

171. A controlled release formulation comprising a drug and a sufficient amount of a lacquer agent to provide a controlled release of the drug.

172. The formulation of claim 171 wherein said lacquer agent is selected from the group consisting of corn oil, cottonseed oil, menhaden oil, pine oil, peanut oil, safflower oil, sesame oil, soybean oil, linseed oil and mixtures thereof.

173. The formulation of claim 171 wherein said lacquer agent is selected from the group consisting of fatty acids of C8-C20 oils which can be saturated, unsaturated, glycerides thereof, and combination thereof.

174. The formulation of claim 171 wherein said lacquer agent is selected from the group consisting of branched or polycarboxylated oils such as linoleic acid, linolenic acid, oleic acid and combinations thereof.

175. The formulation of claim 171 wherein said lacquer agent is selected from the group consisting of caprylic acid, capric acid, lauric acid, myristic acid, palmitic acid, margaric acid, stearic acid, arachidic acid, behenic acid, lignoceric acid and combinations thereof.

176. The formulation of claim 171 wherein said lacquer agent is at least partially interdispersed with said drug.

177. The formulation of claim 171 wherein said lacquer agent is coated onto said drug.

178. The formulation of claim 171 wherein said formulation is in multiparticulate form.

179. The formulation of claim 171 wherein said formulation is a tablet.

180. The formulation of claim 171 further comprising a channeling agent such as polyvinylpyrrolidone, polyethyleneglycols, dextrose, sucrose, mannitol, xylitol, lactose and combinations thereof.

181. The formulation of claim 171 further comprising a dispersing agent such as colloidal silicone dioxide, talc, kaolin, silicone dioxide, colloidal calcium carbonate, bentonite, Fuller's earth, magnesium aluminum silicate and mixtures thereof.

182. A method of preparing a multiparticulate drug formulation for gastrointestinal deposition comprising preparing a non-compressed free flowing plurality of particles comprising a drug and air jet sieving said particles to separate fine particles.

183. The method of claim 182 wherein said fine particles are less than about 50 micrometers.

184. The method of claim 182 wherein said fine particles are less than about 25 micrometers.

185. The method of claim 182 wherein said fine particles are less than about 10 micrometers.

186. The method of claim 182 further comprising filtering said particles prior to air jet sieving to remove particles greater than about 500 micrometers.

187. The method of claim 182 further comprising filtering said particles prior to air jet sieving to remove particles greater than about 750 micrometers.

188. The method of claim 182 further comprising filtering said particles prior to air jet sieving to remove particles greater than about 1 mm.

189. The method of claim 182 further comprising placing a plurality of said multiparticulates in a dosing device capable of metering a unit dose of said formulation for oral delivery.

190. A composition obtained from a method of claim 182.

191. A formulation for gastrointestinal deposition comprising a non-compressed free flowing plurality of particles comprising a core comprising chlorpheniramine or a salt thereof and a pharmaceutically acceptable excipient, said core overcoated with a functional coating, said particles having a mean diameter of greater than 10 μm to about 1 mm.
